(19) United States
(12) Patent Application Publication
(10) Pub. No.: US 2022/0259291 A1
(43) Pub. Date: Aug. 18, 2022

(54) ANTIBODY PURIFICATION METHODS AND COMPOSITIONS THEREOF

(71) Applicant: Takeda Pharmaceutical Company LImited, Osaka (JP)

(72) Inventors: Sheldon F. OPPENHEIM, Wellesley Hills, MA (US); George Parks, Wakefield, MA (US); Norbert Schuelke, East Walpole, MA (US); Lulfiye Kurt, Cambridge, MA (US); Michael E. Dolan, Watertown, MA (US)

(21) Appl. No.: 17/596,422

(22) PCT Filed: Jun. 10, 2020
(86) PCT No.: PCT/US2020/037069

§ 371 Date: Dec. 9, 2021

Related U.S. Application Data
(60) Provisional application No. 62/859,580, filed on Jun. 10, 2019.

(51) Int. Cl. C07K 16/06; C07K 1/22; C07K 1/18; C07K 1/16

(52) U.S. Cl. CPC C07K 16/065; C07K 1/165; C07K 1/18; C07K 1/22

(57) ABSTRACT

Methods of purifying a humanized α4β7 antibody, such as vedolizumab, produced in a mammalian cell culture are described herein, as are compositions resulting from said purification processes.

### RELATED APPLICATIONS

This application is a U.S. National Phase Application under 35 U.S.C. § 371 of International Appln. No. PCT/US2020/037069, filed on Jun. 10, 2020, which claims priority to U.S. Provisional Application No. 62/859,580 filed on Jun. 10, 2019. The entire contents of the foregoing applications are incorporated herein by reference.

### FIELD OF THE INVENTION

The present invention relates to methods for purifying an anti-α4β7 antibody, or a fragment thereof.

### SEQUENCE LISTING

This application contains a Sequence Listing which has been submitted electronically in ASCII format and is hereby incorporated by reference in its entirety. Said ASCII copy, created on Jun. 5, 2020, is named T103022_1120US_SL.txt and is 10,015 bytes in size. The entire contents of the Sequence Listing in the sequence listing.txt file are incorporated herein.

### BACKGROUND

Large-scale, economic purification of proteins is an increasingly important concern in the biotechnology industry. Generally, biologic medicines are produced by cell culture using prokaryotic, e.g., bacterial, or eukaryotic, e.g., mammalian or fungal, cell lines that have been engineered to produce the therapeutic protein of interest in large quantities. Since the cell lines used are living organisms, they must be fed a complex cell culture medium comprising sugars, amino acids, and growth factors, sometimes supplied from preparations of animal serum. Separation of the desired recombinant therapeutic protein from process-related impurities, including, for example, cell culture media components, host cell proteins (HCPs), host nucleic acids, and/or chromatographic materials, as well as product-related impurities such as aggregates, mis-folded species, or fragments of the protein of interest, to a purity sufficient for use as a human therapeutic poses a formidable challenge.

Product-related and process-related impurities, including aggregates, have the potential to interfere with the purification process, affect the protein during storage, and/or can potentially be a cause of adverse reactions upon administration of an antibody to a subject as a pharmaceutical (Shukla et al., J. Chromatogr. B. Analyt. Technol. Biomed. Life Sci., 848(1), 28-39).

Accordingly, there remains a need in the art for improved methods of purification of therapeutic proteins, e.g., antibodies, to high purity, while removing impurities effectively, improving the recovery rate of the protein, and maintaining therapeutic requirements.

### SUMMARY OF THE INVENTION

The present invention provides, inter alia, methods for purifying an anti-α4β7 antibody, such as vedolizumab, e.g., from a liquid solution.

In one aspect, the invention features a method for obtaining a composition comprising an anti-α4β7 antibody from a liquid solution comprising an anti-α4β7 antibody and one or more impurities, said method comprising contacting a matrix comprising Protein A with the liquid solution comprising the anti-α4β7 antibody and one or more impurities, such that the anti-α4β7 antibody binds to the Protein A; washing the matrix comprising Protein A with a wash solution; and eluting the anti-α4β7 antibody from the matrix comprising Protein A by contacting the matrix with an elution solution having a pH of 3.2 to 4, such that a composition comprising the anti-α4β7 antibody is obtained, wherein the anti-α4β7 antibody is a humanized antibody, is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In one embodiment, the method is used for obtaining a composition comprising less than 1% high molecular weight (HMW) aggregate from a liquid solution comprising the anti- α4β7 antibody and one or more impurities, said method comprising contacting a matrix comprising Protein A with the liquid solution comprising the anti-α4β7 antibody and one or more impurities, such that the anti-α4β7 antibody binds to the Protein A; said washing of the matrix comprising Protein A with a wash solution; and said eluting of the anti-α4β7 antibody from the matrix comprising Protein A by contacting the matrix with an elution solution having a pH of 3.2 to 4, such that a composition comprising less than 1% HMW aggregate is obtained.

In one embodiment, the Protein A is immobilized on a solid phase. In one embodiment, the solid phase comprises one or more of a bead, a gel, and a resin.

In one embodiment, the wash solution has a pH of about 7. In one embodiment, the elution solution comprises citric acid.

In one embodiment, the elution solution has a pH of 3.2 to 3.7.

In another aspect, the invention features a method for obtaining a composition comprising an anti-α4β7 antibody from a liquid solution comprising an anti-α4β7 antibody and one or more impurities, said method comprising contacting a solution comprising an anti-α4β7 antibody and at least one impurity with a hydrophobic interaction chromatography (HIC) resin under conditions that allow flow through of the anti-α4β7 antibody through the HIC resin, such that a composition comprising the anti-α4β7 antibody is obtained, wherein the HIC resin is characterized as a high hydrophobic HIC resin, wherein the anti-α4β7 antibody is a humanized antibody, is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In one embodiment, the method is for obtaining a composition comprising the anti-α4β7 antibody and less than 0.6% HMW aggregate from a liquid solution comprising an anti-α4β7 antibody and one or more impurities, said method comprising said contacting of a solution comprising an anti-α4β7 antibody and at least one impurity with a HIC resin under conditions that allow flow through of the anti-α4β7 antibody through the HIC resin, such that a composition comprising the anti-α4β7 antibody and less than 0.6% HMW aggregate is obtained, wherein the anti-α4β7 antibody is a humanized antibody, is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

In one embodiment, the HIC resin is equilibrated with a phosphate buffer having a pH of less than about 7.2. In one embodiment, the phosphate buffer comprises about 0.35 mM to about 0.15 mM potassium phosphate.

In one embodiment, the resin load is about 55 to 75 mg/ml.

In one embodiment, the composition comprises less than about 0.22 ppm residual protein A.

In one embodiment, the composition contains less than about 0.3 ppm host cell protein (HCP).

In one embodiment, the high hydrophobic HIC resin has a mean pore size of about 50 to 150 μm.

In one embodiment, the high hydrophobic HIC resin has a mean pore size of about 100 nm and/or a pore size of about 100 μm.

In another aspect, the invention features a method for producing a preparation comprising an anti-α4β7 antibody from a liquid solution comprising an anti-α4β7 antibody and one or more impurities, said method comprising contacting the liquid solution comprising the anti-α4β7 antibody and one or more impurities with a mixed mode chromatography resin, such that the anti-α4β7 antibody binds to the resin; washing the mixed mode chromatography resin with a wash solution; and eluting the anti-α4β7 antibody from the mixed mode chromatography resin by contacting the resin with an elution solution having a pH at or above pH 3.9, such that a preparation comprising a purified anti-α4β7 antibody is obtained, wherein the anti-α4β7 antibody comprises a heavy chain variable region set forth in SEQ ID NO:1, and a light chain variable region set forth in SEQ ID NO:2.

In one embodiment of the aforementioned aspect, the method is for obtaining a preparation comprising less than 1% HMW aggregate from a liquid solution comprising the anti-α4β7 antibody and one or more impurities, said method comprising contacting the liquid solution comprising the anti-α4β7 antibody and one or more impurities with a mixed mode chromatography resin, such that the anti-α4β7 antibody binds to the resin; said washing of the mixed mode chromatography resin with a wash solution; and said eluting of the anti-α4β7 antibody from the mixed mode chromatography resin by contacting the resin with an elution solution having a pH at or above pH 3.9, such that a preparation comprising less than 1% HMW aggregate is obtained.

In one embodiment, the elution solution has a pH at or above pH 4.1. In another embodiment, the elution solution has a pH of about pH 3.9 to about pH 4.4.

In some embodiments, the elution solution has a conductivity of 30 mS/cm or less. In certain embodiments, the elution solution has a conductivity of about 20 mS/cm to about 30 mS/cm.

In some embodiments, the elution solution comprises NaCl at a concentration of about 160 mM to about 240 mM.

In certain embodiments, the mixed mode chromatography resin is Capto Adhere ImpRes.

In some embodiments of the above aspect, the method further comprises purifying the anti-α4β7 antibody using a cation exchange (CEX) resin. In some such embodiments, the CEX resin is operated in bind/elute mode.

In another aspect, the invention features a method for producing a preparation comprising an anti-α4β7 antibody from a liquid solution comprising an anti-α4β7 antibody and one or more impurities, said method comprising contacting the liquid solution comprising the anti-α4β7 antibody and one or more impurities with a mixed mode chromatography resin, such that the anti-α4β7 antibody binds to the resin; washing the mixed mode chromatography resin with a wash solution; and eluting the anti-α4β7 antibody from the mixed mode chromatography resin by contacting the resin with an elution solution having a pH at or below pH 4.2 and a conductivity at or below 28 mS/cm, such that a preparation comprising a purified anti-α4β7 antibody is obtained, wherein the anti-α4β7 antibody comprises a heavy chain variable region set forth in SEQ ID NO:1, and a light chain variable region set forth in SEQ ID NO:2.

In some embodiments of the above aspect, the method is for obtaining a preparation comprising an increased yield of an anti-α4β7 antibody from a liquid solution comprising the anti-α4β7 antibody and one or more impurities, said method comprising said contacting of the liquid solution comprising the anti-α4β7 antibody and one or more impurities with a mixed mode chromatography resin, such that the anti-α4β7 antibody binds to the resin; said washing of the mixed mode chromatography resin with a wash solution; and said eluting of the anti-α4β7 antibody from the mixed mode chromatography resin by contacting the resin with an elution solution having a pH at or below pH 4.2 and a conductivity at or below 28 mS/cm, such that a preparation comprising an increased yield of the anti-α4β7 antibody is obtained.

In some embodiments, the elution solution has a pH at or below 4.0. In other embodiments, the elution solution has a pH of about pH 4.2 to about pH 3.8.

In some embodiments, the elution solution has a conductivity of about 18 mS/cm to about 28 mS/cm.

In some embodiments, the elution solution comprises NaCl at a concentration of about 160 mM to about 240 mM.

In some embodiments of the above aspect, the mixed mode chromatography resin is contacted with at least 55 g of the anti-α4β7 antibody per liter of resin. In certain embodiments, the mixed mode chromatography resin is contacted with about 55 g to about 80 g of the anti-α4β7 antibody per liter of resin.

In some embodiments of the above aspect, the mixed mode chromatography resin is Capto Adhere ImpRes.

In some embodiments of the above aspect, the method further comprises purifying the anti-α4β7 antibody using a cation exchange (CEX) resin. In some such embodiments, the CEX resin is operated in bind/elute mode.

In another aspect, the invention features a method for producing a preparation comprising an anti-α4β7 antibody from a liquid solution comprising an anti-α4β7 antibody and one or more impurities, said method comprising contacting the liquid solution comprising the anti-α4β7 antibody and one or more impurities with a cation exchange (CEX) resin, such that the anti-α4β7 antibody binds to the resin; washing the CEX resin with a wash solution; and eluting the anti-α4β7 antibody from the CEX resin by contacting the resin with an elution solution having a conductivity at or below 16 mS/cm, such that a preparation comprising a purified anti-α4β7 antibody is obtained, wherein the anti-α4β7 antibody comprises a heavy chain variable region set forth in SEQ ID NO:1, and a light chain variable region set forth in SEQ ID NO:2.

In some embodiments of the aforementioned aspect, the method is for obtaining a preparation comprising a reduced level of HMW aggregate from a liquid solution comprising the anti-α4β7 antibody and one or more impurities, said method comprising said contacting of the liquid solution comprising the anti-α4β7 antibody and one or more impurities with a CEX resin, such that the anti-α4β7 antibody binds to the resin; said washing of the CEX resin with a wash solution; and said eluting of the anti-α4β7 antibody from the CEX resin by contacting the resin with an elution solution having a conductivity at or below 16 mS/cm, such that a preparation comprising reduced level of HMW aggregate is obtained.

In some embodiments of the above aspect, the elution solution has a conductivity at or below 14 mS/cm. In other embodiments, the elution solution has a conductivity of about 11-16 mS/cm. In yet other embodiments, the elution solution has a conductivity of about 12-14 mS/cm.

In some embodiments of the above aspect, the elution solution comprises NaCl at a concentration of about 70 mM to about 110 mM.

In some embodiments of the above aspect, the elution solution has a pH from about pH 5 to about pH 6. In certain embodiments, the elution solution has a pH from about pH 5.1 to about pH 5.8.

In some embodiments of the above aspect, the anti-α4β7 antibody is loaded on the CEX resin at a concentration of about 25-70 g antibody per liter of resin. In certain embodiments, the anti-α4β7 antibody is loaded on the CEX resin at a concentration of about 30-60 g antibody per liter of resin.

In some embodiments of the above aspect, the CEX resin is Nuvia HR-S.

In some embodiments of the above aspect, the method further comprises purifying the anti-α4β7 antibody using a mixed mode chromatography resin. In some such embodiments, the mixed mode chromatography resin is operated in bind/elute mode.

In another aspect, the invention features a method for producing a preparation comprising an anti-α4β7 antibody from a liquid solution comprising a major isoform of the anti-α4β7 antibody and one or more basic isoform species, the method comprising contacting the liquid solution comprising the anti-α4β7 antibody and one or more basic isoform species with a cation exchange (CEX) resin, such that the anti-α4β7 antibody binds to the resin; washing the CEX resin with a wash solution; and eluting the anti-α4β7 antibody from the CEX resin by contacting the resin with an elution solution having a conductivity at or above 11 mS/cm, such that a preparation comprising a purified anti-α4β7 antibody is obtained, wherein the anti-α4β7 antibody comprises a heavy chain variable region set forth in SEQ ID NO:1, and a light chain variable region set forth in SEQ ID NO:2.

In some embodiments of the aforementioned aspect, the method is for obtaining a preparation comprising a reduced level of basic isoform species of an α4β7 antibody from a liquid solution comprising a major isoform of the anti-α4β7 antibody and one or more basic isoform species, said method comprising said contacting of the liquid solution comprising the anti-α4β7 antibody and one or more basic isoform species with a CEX resin, such that the anti-α4β7 antibody binds to the resin; said washing of the CEX resin with a wash solution; and said eluting of the anti-α4β7 antibody from the CEX resin by contacting the resin with an elution solution having a conductivity at or above 11 mS/cm, such that a preparation comprising a reduced level of basic isoform species is obtained.

In one embodiment, the purified composition comprises about 4% to about 20% basic isoform.

In some embodiments of the above aspect, the elution solution has a conductivity at or above 12 mS/cm. In other embodiments, the elution solution has a conductivity of about 11-16 mS/cm. In yet other embodiments, the elution solution has a conductivity of about 12-14 mS/cm.

In some embodiments of the above aspect, the elution solution has a pH from about pH 5 to about pH 6. In certain embodiments, the elution solution has a pH from about pH 5.1 to about pH 5.8.

In some embodiments of the above aspect, the anti-α4β7 antibody is loaded on the CEX resin at a concentration of about 25-70 g antibody per liter of resin. In certain embodiments, the anti-α4β7 antibody is loaded on the CEX resin at a concentration of about 30-60 g antibody per liter of resin.

In some embodiments, the elution solution comprises sodium chloride, e.g., 70 to 110 mM sodium chloride.

In some embodiments of the above aspect, the CEX resin is Nuvia HR-S.

In some embodiments of the above aspect, the method further comprises purifying the anti-α4β7 antibody using a mixed mode chromatography resin. In some such embodiments, the mixed mode chromatography resin is operated in bind/elute mode.

In one embodiment of any of the aforementioned aspects, the antibody was produced in a Chinese Hamster Ovary (CHO) host cell.

In one embodiment, the host cell is a GS-CHO cell.

In one embodiment of any of the aforementioned aspects, the anti-α4β7 antibody comprises a heavy chain variable region sequence as set forth in SEQ ID NO: 1, and a light chain variable region sequence as set forth in SEQ ID NO: 5.

In one embodiment of any of the above aspects, the anti-α4β7 antibody is vedolizumab.

Additionally, the invention also comprises the following embodiments:

1. A method for obtaining a composition comprising less than 1% HMW aggregate from a liquid solution comprising an anti-α4β7 antibody and one or more impurities, said method comprising

contacting a matrix comprising Protein A with the liquid solution comprising the anti-α4β7 antibody and one or more impurities, such that the anti-α4β7 antibody binds to the Protein A;

washing the matrix comprising Protein A with a wash solution; and eluting the anti-α4β7 antibody from the matrix comprising Protein A by contacting the matrix with an elution solution having a pH of 3.2 to 4, such that a composition comprising less than 1% HMW aggregate is obtained,

wherein the anti-α4β7 antibody is a humanized antibody, is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

2. The method of item 1, wherein the Protein A is immobilized on a solid phase.

3. The method of item 2, wherein the solid phase comprises one or more of a bead, a gel, and a resin.

4. The method of any one of items 1 to 3, wherein the wash solution has a pH of about 7.

5. The method of any one of items 1 to 4, wherein the elution solution comprises citric acid.

6. The method of any one of items 1 to 5, wherein the elution solution has a pH of 3.2 to 3.7.

7. A method for obtaining a composition comprising an anti-α4β7 antibody and less than 0.6% HMW aggregate from a liquid solution comprising an anti-α4β7 antibody and one or more impurities, said method comprising

contacting a solution comprising an anti-α4β7 antibody and at least one impurity with a hydrophobic interaction chromatography resin (HIC) resin under conditions that allow flow through of the anti-α4β7 antibody through the HIC resin, such that a composition comprising the anti-α4β7 antibody and less than 0.6% HMW aggregate is obtained,

wherein the HIC resin is characterized as a high hydrophobic HIC resin,

wherein the anti-α4β7 antibody is a humanized antibody, is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

8. The method of item 7, wherein the HIC resin is equilibrated with a phosphate buffer having a pH of less than about 7.2.

9. The method of item 8, wherein the phosphate buffer comprises about 0.35 mM to about 0.15 mM potassium phosphate.

10. The method of any one of items 7 to 9, wherein the resin load is about 55 to 75 mg/ml.

11. The method of any one of items 7 to 10, wherein the composition comprises less than about 0.22 ppm residual protein A.

12. The method of any one of items 7 to 11, wherein the composition contains less than about 0.3 ppm host cell protein (HCP).

13. The method of any one of items 7 to 12, wherein the high hydrophobic HIC resin has a mean pore size of about 50 to 150 μm.

14. The method of any one of items 7 to 12, wherein the high hydrophobic HIC resin has a mean pore size of about 100 nm and/or a pore size of about 100 μm.

15. The method of any one of items 1 to 14, wherein the antibody was produced in a Chinese Hamster Ovary (CHO) cell.

16. The method of item 15, wherein the host cell is a GS-CHO cell.

17. The method of any one of item 1 to 16, wherein the anti-α4β7 antibody comprises a heavy chain variable region sequence as set forth in SEQ ID NO: 1, and a light chain variable region sequence as set forth in SEQ ID NO: 5.

18. The method of any one of item 1 to 16, wherein the anti-α4β7 antibody is vedolizumab.

### BRIEF DESCRIPTION OF THE DRAWINGS

FIG. 1 depicts aggregates as a function of Protein A eluate pH.

FIG. 2 is a plot depicting the results of a prediction profiler assay, screening for performance outputs.

FIG. 3 graphically depicts a comparison of vedolizumab vs. three other IgG antibodies and the pH characteristics for eluting each antibody from a cation exchange column.

FIG. 4 is a linear regression model surface plot depicting Capto Adhere ImpRes step recovery versus elution buffer pH and conductivity.

FIG. 5 is a linear regression model surface plot depicting Capto Adhere ImpRes step recovery versus elution buffer pH and load amount.

FIG. 6 is a linear regression model surface plot depicting Capto Adhere ImpRes % HMW species versus elution buffer pH and conductivity.

FIG. 7 is a linear regression model surface plot depicting Capto Adhere ImpRes % HMW species versus elution buffer pH and load amount.

FIG. 8 is a HMW surface plot depicting the effect of elution buffer pH and elution buffer conductivity on % HMW species.

FIG. 9 is a HMW clearance surface plot depicting the effect of elution buffer pH and elution buffer conductivity on HMW clearance.

FIG. 10 is a monomer surface plot depicting the effect of elution buffer pH and elution buffer conductivity on % monomer species.

FIG. 11 is an acidic surface plot depicting the effect of elution buffer pH and elution buffer conductivity on % acidic isoform species.

FIG. 12 is a major surface plot depicting the effect of elution buffer pH and elution buffer conductivity on % major isoform species.

FIG. 13 is a basic surface plot depicting the effect of elution buffer pH and elution buffer conductivity on % basic isoform species.

### DETAILED DESCRIPTION OF THE INVENTION

The instant invention relates, inter alia, to purification methods for controlling the amount of product-related substances (e.g., aggregates, such as high molecular weight (HMW) aggregates, mis-folded species, or protein fragments) and/or process-related impurities (e.g., host cell proteins (HCPs), host cell nucleic acids, viruses, chromatographic materials, and/or media components) present in purified preparations of an anti-α4β7 antibody or antigen-binding fragment thereof, e.g., vedolizumab.

### I. Definitions

In order that the present invention may be more readily understood, certain terms are first defined.

The cell surface molecule, “α4β7 integrin,” or “α4β7” (used interchangeably throughout) is a heterodimer of an α4 chain (CD49D, ITGA4) and a δ7 chain (ITGB7). Human α4-integrin and β7-integrin genes GenBank (National Center for Biotechnology Information, Bethesda, Md.) RefSeq Accession numbers NM_000885 and NM_000889, respectively) are expressed by B and T lymphocytes, particularly memory CD4+ lymphocytes. Typical of many integrins, α4β7 can exist in either a resting or activated state. Ligands for α4β7 include vascular cell adhesion molecule (VCAM), fibronectin and mucosal addressin (MAdCAM (e.g., MAdCAM-1)). An antibody that binds to α4β7 integrin is referred to herein as an “anti-α4β7 antibody”.

As used herein, an antibody, or antigen-binding fragment thereof, that has “binding specificity for the α4β7 complex” binds to α4β7, but not to α4β7 or α_{E}B7. Vedolizumab is an example of an antibody that has binding specificity for the α4β7 complex.

The term “antibody” as used herein, is intended to refer to an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the antibody has a fragment crystallizable (Fc) region. In certain embodiments, the antibody is an IgG1 isotype and has a kappa light chain.

A “CDR” or “complementarity determining region” is a region of hypervariability interspersed within regions that are more conserved, termed “framework regions” (FR). As used herein, the term “antigen binding fragment” or “antigen binding portion” of an antibody refers to Fab, Fab′, F(ab′)₂, and Fv fragments, single chain antibodies, functional heavy chain antibodies (nanobodies), as well as any portion of an antibody having specificity toward at least one desired epitope, that competes with the intact antibody for specific binding (e.g., an isolated portion of a complementarity determining region having sufficient framework sequences so as to bind specifically to an epitope). Antigen binding fragments can be produced by recombinant techniques, or by enzymatic or chemical cleavage of an antibody.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable CDR loops correspond to those of a non-human antibody and all or substantially all of the FRs are those of a human antibody sequence. The humanized antibody optionally also will comprise at least a portion of an antibody constant region (Fc), typically that of a human antibody. For further details, see Jones et al., Nature 321:522-525 (1986); Riechmann et al., Nature 332:323-329 (1988); and Presta, Curr. Op. Struct. Biol. 2:593-596 (1992).

As used herein, the term “recombinant antibody” refers to an antibody produced as the result of the transcription and translation of a gene(s) carried on a recombinant expression vector(s) that has been introduced into a host cell, e.g. a mammalian host cell. In certain embodiments the recombinant protein is an antibody of an isotype selected from group consisting of: IgG (e.g., IgG1, IgG2, IgG3, IgG4), IgM, IgA1, IgA2, IgD, or IgE. In certain embodiments the recombinant antibody is an IgG1.

The term “recombinant host cell” (used interchangeably herein with the term “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Further, it should be understood that unless specified otherwise, where the term “cell” is used, e.g., host cell or mammalian cell or mammalian host cell, it is intended to include a population of cells.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the term “upstream process” in the context of protein, e.g., antibody, preparation, refers to activities involving the production and collection of proteins (e.g. antibodies) from host cells (e.g., upon cell culture to produce a protein of interest, e.g., antibody).

As used herein, the term “downstream process” refers to one or more techniques used after the upstream process to purify the protein, e.g., antibody, of interest. For example, a downstream process technique includes purification of the protein product, using, for example, affinity chromatography, including Protein A affinity chromatography, ion exchange chromatography, such as anion or cation exchange chromatography, size exclusion chromatography, mixed mode chromatography, hydrophobic interaction chromatography (HIC), or displacement chromatography.

As used herein, the terms “culture” and “cell culture” generally refer to the process by which cells are grown under controlled conditions, generally outside of their natural environment. “Culturing” a cell refers to contacting a cell with a cell culture medium under conditions suitable to the survival and/or growth and/or proliferation of the cell. Cell culture, in certain embodiments, refers to methods for generating and maintaining a population of host cells capable of producing a recombinant protein of interest, e.g., an anti-α4β7 antibody, as well as the methods and techniques for the production and collection of the protein of interest. For example, once an expression vector has been incorporated into an appropriate host, e.g., a host cell in culture, the host can be maintained under conditions suitable for expression of the relevant nucleotide coding sequences, and the collection and purification of the desired recombinant protein. “Cell culture” can also refer to a solution containing cells.

The term “clarified harvest,” as used herein, refers to a liquid material containing a protein of interest, for example, an anti-α4β7 antibody, that has been extracted from cell culture, for example, a fermentation bioreactor, after undergoing one or more process steps to remove solid particles, such as cell debris and particulate impurities from the material. Following cell culture, the harvest is typically purified to remove cells and cellular debris using separation techniques, such as centrifugation and filtration. Initial clarification, particulate removal steps result in a “clarified harvest” that can be used, for example, in subsequent chromatographic steps (downstream processing). The clarified harvest is generally the starting material for downstream processing, such as downstream processing steps described herein.

A “chromatographic support”, as used herein, refers to a solid or porous matrix of a specific chemical composition or specific three-dimensional structure or on which specific chemical groups or macromolecules may be immobilized in order to perform chromatography, including affinity chromatography, gel filtration (size exclusion chromatography), or ion exchange chromatography. Examples of a chromatographic support include, but are not limited to, resin (e.g., agarose) or a membrane. A “chromatographic housing,” as used herein refers to a structure containing the chromatographic support. Examples of a chromatographic housing include a column or a cartridge, or other container.

The term “buffer,” as used herein, refers to an aqueous solution that resists changes in pH by the action of its acid-base conjugate components. A buffer is used to establish a specified set of conditions to mediate control of a processing step or chromatographic support, such as a chromatography resin or membrane.

The term “equilibration solution,” as used herein, refers to an aqueous liquid formulated to create the initial operating conditions for a processing step or chromatographic support, such as a chromatographic operation. An equilibration solution is used to prepare, for example, a solid phase, e.g., a chromatographic support, e.g., resin or membrane, for loading the protein, e.g., antibody, of interest.

The term “wash” or “wash solution,” as used herein, refers to an aqueous liquid formulated to displace unbound contaminants from a chromatographic support, such as resin or membrane. In some embodiments, a wash is passed over a solid support, e.g., a resin or membrane, following loading with a protein, e.g., antibody, of interest and prior to elution of the protein, e.g., antibody, of interest. In one embodiment, a wash has biochemical characteristics similar to the equilibration solution.

A “flow-through operation,” as used herein, refers to a process by which the protein does not substantially bind to a matrix, e.g., a hydrophobic chromatography resin, and/or elutes during a wash, while impurities remain associated with the chromatographic support.

The term “elution solution” or “eluent,” as used herein, refers to an aqueous liquid formulated to displace a protein of interest, e.g., antibody from a chromatographic support, e.g., resin or membrane. In one embodiment, an elution solution has biochemical characteristics different from the equilibration and/or wash solution, such that the protein, e.g., antibody, of interest prefers to associate with the elution solution, rather than with the chromatographic support, e.g., resin or membrane.

The term “impurity,” as used herein with respect to impurities contained in a solution comprising an antibody to be purified, includes both process-related impurities and product-related impurities.

The term “process-related impurity,” as used herein, refers to an impurity (or impurities) that are present in a composition, e.g., a solution, comprising a protein but are not derived from the protein itself. For example, process-related impurities include, but are not limited to, cell culture media components, host cell components (such as proteins (HCPs), host cell nucleic acids, or lipid-containing subcellular structures or fragments thereof), viruses, trace metals or ions from the buffers, leachable materials from the material-handling vessels or chromatographic support. Process-related impurities can be formed during the preparation (upstream and/or downstream processing) of the protein, e.g., the antibody.

As used herein, the term “host cell impurity” refers to any proteinaceous, nucleic acid contaminant, lipid contaminant, or by-product introduced by the host cell line, cell cultured fluid, or cell culture. Examples of impurities include, but are not limited to, Chinese Hamster Ovary Protein (CHOP), E. coli protein, yeast protein, simian COS protein, or myeloma cell protein (e.g., NSO protein (mouse plastocytoma cells derived from a BALB/c mouse)).

The term “product-related impurities,” as used herein, includes impurities derived from the protein, e.g., antibody, of interest itself. For example, product-related impurities include, but are not limited to, aggregates (e.g., HMW), mis-folded species, oxidized or deamidated species or low molecular weight fragments, of the antibody of interest.

As used herein, the terms “aggregate” or “aggregates” refer to the association of two or more antibodies or antibody fragments. For example, an aggregate can be a dimer, trimer, tetramer, or a multimer greater than a tetramer, of antibodies and/or antibody fragments. Antibody aggregates can be soluble or insoluble. The association between the aggregated molecules may be either covalent or non-covalent without respect to the mechanism by which they are associated. The association may be direct between the aggregated molecules or indirect through other molecules that link them together. Examples of the latter include, but are not limited to disulfide linkages with other proteins, hydrophobic associations with lipids, charge associations with DNA, affinity associations with leached protein A, or mixed mode associations with multiple components. Aggregates can be irreversibly formed either during protein expression in cell culture, during protein purification in downstream processing, or during storage of the drug product. The presence of aggregates in a solution can be determined using, for example, size exclusion chromatography (SEC) (e.g., SEC with UV detection, SEC with light scattering detection (SEC-LSD)), field flow fractionation, analytical ultracentrifugation sedimentation velocity, or capillary electrophoresis-sodium dodecyl sulfate (CE-SDS, reduced and non-reduced).

The term “high molecular weight” or “HMW” is used to indicate an antibody complex having a molecular weight greater than a monomer antibody. In one embodiment, a HMW aggregate has a molecular weight greater than about 147 kDa. The presence of high molecular weight aggregates may be determined by standard methods known in the art, e.g., size-exclusion chromatography (SEC).

“Substantially purified” with regard to the desired protein means that the purified sample comprising the protein comprises at least 90%, at least 91%, at least 92%, at least 93%, at least 94%, at least 95%, at least 96%, at least 97%, at least 97.5%, at least 98%, at least 98.5%, or at least 99% of the desired recombinant protein with less than 3%, less than 2.5%, less than 2%, less than 1.5%, less than 1%, or less than 0.5% of impurities.

The term “about” denotes that the thereafter following value is no exact value but is the center point of a range that is +/−5% of the value of the value. If the value is a relative value given in percentages the term “about” also denotes that the thereafter following value is no exact value but is the center point of a range that is +/−5% of the value, whereby the upper limit of the range cannot exceed a value of 100%.

### II. Methods and Compositions Relating to Antibody Purification

Provided herein are methods for purifying an anti-α4β7 antibody, such as vedolizumab, e.g., from a liquid solution, e.g., from a mammalian cell culture clarified harvest. The invention is based, at least in part, on certain aspects of the antibody purification process which reduce the level of impurities, including, for example process-related impurities, such as cell culture media components, host cell proteins (HCPs), host cell nucleic acids, viruses, and chromatographic materials, and product-related impurities, such as aggregates (including HMW aggregates), mis-folded species, and fragments of the antibody of interest, that are present in the antibody solution. The methods of the invention are useful for purifying an anti-α4β7 antibody, particularly vedolizumab or an antibody having the binding regions, i.e., CDRs or variable regions, of vedolizumab, such that the antibody can be formulated for use in human patients.

In particular, the methods disclosed herein are useful for achieving low levels of antibody aggregation, e.g., HMW antibody aggregates. In certain embodiments, the methods disclosed herein provide compositions having about 0% to 5.0% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, or 5%) aggregates, e.g., HMW aggregates. In particular embodiments, the methods disclosed herein provide compositions having about 0% to 2%, ≤2%, ≤1.9%, ≤1.8%, ≤1.7%, ≤1.6%, ≤1.5%, ≤1.4%, ≤1.3%, ≤1.2%, ≤1.1%, ≤1%, ≤0.9%, ≤0.8%, ≤0.7%, ≤0.6% or ≤0.5% aggregates, e.g., HMW aggregates. Also included in the invention are compositions comprising an anti-α4β7 antibody and said low levels of HMW aggregate.

In particular, the methods disclosed herein may be used to produce the anti-α4β7 antibody vedolizumab, or antibodies having antigen binding regions of vedolizumab. Vedolizumab is also known by its trade name ENTYVIO® (Takeda Pharmaceuticals, Inc.). Vedolizumab is a humanized antibody that comprises a human IgG1 framework and constant regions and antigen-binding CDRs from the murine antibody Act-1. The vedolizumab CDRs, variable regions and mutated Fc region (mutated to eliminate Fc effector functions) are described in U.S. Pat. No. 7,147,851, incorporated by reference herein).

Vedolizumab is a humanized monoclonal antibody that specifically binds to the α4β7 integrin, e.g., the α4β7 complex, and blocks the interaction of α4β7 integrin with mucosal addressin cell adhesion molecule-1 (MAdCAM-1) and inhibits the migration of memory T-lymphocytes across the endothelium into inflamed gastrointestinal parenchymal tissue. Vedolizumab does not bind to or inhibit function of the α4β1 and αEβ7 integrins and does not antagonize the interaction of α4 integrins with vascular cell adhesion molecule-1 (VCAM-1).

The α4β7 integrin is expressed on the surface of a discrete subset of memory T-lymphocytes that preferentially migrate into the gastrointestinal tract. MAdCAM-1 is mainly expressed on gut endothelial cells and plays a critical role in the homing of T-lymphocytes to gut lymph tissue. The interaction of the α4β7 integrin with MAdCAM-1 has been implicated as an important contributor to mucosal inflammation, such as the chronic inflammation that is a hallmark of ulcerative colitis and Crohn's disease. Vedolizumab may be used to treat inflammatory bowel disease, including Crohn's disease and ulcerative colitis, pouchitis, including chronic pouchitis, graft-versus host disease, and HIV.

The heavy chain variable region of vedolizumab is provided herein as SEQ ID NO:1, and the light chain variable region of vedolizumab is provided herein as SEQ ID NO:5. Vedolizumab comprises a heavy chain variable region comprising a CDR1 of SEQ ID NO:2, a CDR2 of SEQ ID NO:3, and a CDR3 of SEQ ID NO:4. Vedolizumab comprises a light chain variable region comprising a CDR1 of SEQ ID NO:6, a CDR2 of SEQ ID NO:7 and CDR3 of SEQ ID NO:8. In one embodiment, the antibody comprises a heavy chain comprising the amino acid sequence of SEQ ID NO: 9, and a light chain comprising the amino acid sequence of SEQ ID NO: 10. Vedolizumab and the sequences of vedolizumab are also described in U.S. Patent Publication No. 2014/0341885 and U.S. Patent Publication No. 2014-0377251, the entire contents of each which are expressly incorporated herein by reference in their entireties. The methods disclosed herein can be performed using an antibody comprising binding regions, e.g., CDRs or variable regions, set forth above and in the enclosed sequence table.

Methods of producing antibodies are known in the art. Mammalian host cells are engineered to stably express an anti-α4β7 antibody (e.g., vedolizumab). The overall cell culture process and considerations for production of monoclonal antibodies such as vedolizumab is described in Li et al. (2010) mAbs 2:5, 466-477, and Birch and Racher (2006) Adv. Drug Delivery Rev. 58:671-685, incorporated by reference herein.

When using cell culture techniques, the anti-α4β7 antibody can be produced intracellularly, in the periplasmic space, or directly secreted into the medium. In embodiments where the anti-α4β7 antibody is produced intracellularly, the particulate debris, either host cells or lysed cells (e.g., resulting from homogenization) can be removed by a variety of means, including but not limited to, centrifugation or filtration. Where the anti-α4β7 antibody is secreted into the medium, supernatants from such expression systems can be first concentrated using a commercially available protein concentration filter.

The culture medium or lysate may undergo one or more process steps, such as settling, flocculation, centrifugation, and/or filtration, to remove particulate cell debris to form a clarified cell culture supernatant, or clarified harvest. An antibody, e.g., anti-α4β7 antibody (e.g., vedolizumab or an antibody having binding regions corresponding to vedolizumab) thereafter is purified, as described in detail below, to remove impurities, e.g., process-related impurities, such as cell culture media components, host cell proteins (HCPs), host cell nucleic acids, viruses, and chromatographic materials, and product-related impurities, such as aggregates (including HMW aggregates), mis-folded species, and fragments of the antibody of interest.

The purification process may begin after the antibody has been produced using upstream production methods described above and/or by alternative production methods conventional in the art. Once a clarified solution or mixture comprising the antibody has been obtained, separation of the antibody of interest from process-related impurities, such as the other proteins produced by the cell, as well as product-related substances, is performed. In certain non-limiting embodiments, such separation is performed using CEX, AEX, and/or MM chromatography. In certain embodiments, a combination of one or more different purification techniques, including affinity separation step(s), ion exchange separation step(s), mixed-mode step(s), and/or hydrophobic interaction separation step(s) can also be employed. Such additional purification steps separate mixtures of antibodies on the basis of their charge, degree of hydrophobicity, and/or size. In one aspect of the invention, such additional separation steps are performed using chromatography, including hydrophobic, anionic or cationic interaction (or a combination thereof). Numerous chromatography resins are commercially available for each of these techniques, allowing accurate tailoring of the purification scheme to the particular antibody involved. Each of the separation methods allow antibodies to either traverse at different rates through a column, achieving a physical separation that increases as they pass further through the column, or to adhere selectively to a separation resin (or medium). The antibodies are then differentially eluted using different eluents. In some cases, the antibody of interest is separated from impurities when the impurities specifically adhere to the column's resin and the antibody of interest does not, i.e., the antibody of interest is contained in the flow through, while in other cases the antibody of interest will adhere to the column's resin, while the impurities and/or product-related substances are extruded from the column's resin during a wash cycle, after which the antibody is released by a change of the liquid surrounding the resin and the antibody of interest is eluted from the column.

In certain embodiments, in a “capture step,” a solution comprising an antibody is subjected to affinity chromatography to purify the antibody away from impurities. In certain embodiments, the chromatographic material is capable of selectively or specifically binding to the antibody of interest (“capture”). Non-limiting examples of such chromatographic material include: Protein A, Protein G, chromatographic material comprising, for example, an antigen bound by an antibody of interest, and chromatographic material comprising an Fc binding protein.

In specific embodiments, the affinity chromatography step described herein involves subjecting a clarified harvest comprising an anti-α4β7 antibody to a Protein A matrix, e.g., a chromatography column comprising a Protein A resin. In certain embodiments, Protein A resin is useful for affinity purification and isolation of a variety of antibody isotypes, particularly IgG1, IgG2, and IgG4. Protein A is a bacterial cell wall protein that binds to mammalian IgGs primarily through their Fc regions. In its native state, Protein A has five IgG binding domains as well as other domains of unknown function.

Purification of an Anti-α4β7 Antibody Using a Protein A Resin

In one aspect, the methods described herein comprise the purification of an anti-α4β7 antibody (e.g., vedolizumab) from a liquid solution, e.g., a clarified harvest, comprising the antibody and one or more impurities using Protein A. The method includes binding the anti-α4β7 antibody to an affinity chromatography matrix such as Protein A. In certain embodiments, the antibody solution can be loaded onto the affinity chromatography matrix at more than 10 g/L, such as 10 to 50 g/L, 20 to 45 g/L or 30 to 40 g/L. For example, the antibody solution can be loaded onto a Protein A affinity chromatography matrix at about 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, or 50 g/L.

There are several commercial sources for Protein A resin. One suitable resin is MabSelect™ from GE Healthcare. Suitable resins include, but not limited to, MabSelect SuRe™, MabSelect SuRe LX, MabSelect, MabSelect Xtra, rProtein A Sepharose from GE Healthcare, MabSelect™ ProA resin, ProSep HC, ProSep Ultra, and ProSep Ultra Plus from EMD Millipore, MabCapture from Life Technologies.

The Protein A column can be equilibrated with a suitable equilibration solution prior to sample loading. Following the loading of the column, the column can be washed one or multiple times using a suitable set of solutions, to reduce the one or more impurities, where the anti-α4β7 antibody remains bound to the Protein A.

In some embodiments, the Protein A matrix is washed more than one time. In some embodiments, the Protein A matrix is washed three times. In one embodiment, one or more of the washes comprises phosphate. In one embodiment, the affinity column can be washed with an initial wash solution comprising PBS, followed by a second wash solution comprising NaCl and PBS, followed by a third wash solution comprising PBS. In one embodiment, the first and third wash solutions are the same. In one embodiment, the second wash solution comprises NaCl (e.g., 1 M NaCl) and PBS, and has a pH of 7.2. In another embodiment, one or more of the wash solutions have a pH of about 7.0-7.4. In one embodiment, one or more of the wash solutions have a pH of about 7.2.

In other embodiments, the affinity column is washed with an initial wash solution comprising PBS followed by second and third solutions comprising a buffer, such as citrate, acetate or phosphate. In one embodiment, the second and third solutions comprise a sodium citrate buffer. In one embodiment, the sodium citrate buffers in the second and third wash solutions are the same. In another embodiment, the sodium citrate buffers in the second and third wash solutions are different. In one embodiment, the sodium citrate buffer in the second wash solution has a higher molarity than the sodium citrate buffer in the third wash solution. In an embodiment, the sodium citrate buffer in the second wash solution has a molarity of 75 mM to 125 mM or 100 mM and the sodium citrate buffer in the third wash solution has a molarity of 15 mM to 40 mM or 25 mM. In one embodiment, the pH of the last wash is 5.6 to 6.2. In one embodiment, the elution solution has about the same conductivity as the last wash.

The Protein A column can then be eluted using an appropriate elution solution. For example, glycine-HCL, acetic acid or citric acid can be used as an elution solution. In one embodiment, the elution solution is a citric acid, e.g., sodium citrate, elution solution. In some embodiments, the elution solution can have a pH of about 3.0 to 4.0 (e.g., about 3.1 to 4.0, 3.2 to 4.0, 3.3 to 4.0, 3.4 to 4.0, 3.5 to 4.0, 3.6 to 4.0, 3.7 to 4.0, 3.8 to 4.0, or 3.9 to 4.0). In certain embodiments, the elution solution has a pH of about 3.0 to 3.4. In some embodiments, the elution solution can have a pH of about 3.0, about 3.1, about 3.2, about 3.3, about 3.4, about 3.5, about 3.6, about 3.7, about 3.8, about 3.9, or about 4.0. In some embodiments, the elution solution has a pH at or above 3.3. In some embodiments, the elution solution has a pH at or above 3.4. In some embodiments, the elution solution has a pH at or above 3.5. In some embodiments, the elution solution has a pH at or above 3.6. In some embodiments, the elution solution has a pH at or above 3.7. In some embodiments, the elution solution has a pH at or above 3.8. In some embodiments, the elution solution has a pH at or above 3.9. The eluate can be monitored using techniques well known to those skilled in the art. The eluate fractions of interest can be collected and then prepared for further processing.

As shown in the examples, embodiments in which the Protein A affinity column elution buffer pH has higher pH, e.g., 3.3 to 4.0, than embodiments in which the Protein A affinity column elution buffer pH has lower pH, e.g., 2.9 to 3.3, the eluate comprising the anti-α4β7 antibody has fewer impurities, such as HMW aggregates. In some embodiments, the eluate contains the anti-α4β7 antibody and contains about 0% to 5.0% (e.g., 0-0.1%, 0-0.2%, 0-0.3%, 0-0.4%, 0-0.5%, 0-0.6%, 0-0.7%, 0-0.8%, 0-0.9%, 0-1%, 0-1.1%, 0-1.2%, 0-1.3%, 0-1.4%, 0-1.5%, 0-1.6%, 0-1.7%, 0-1.8%, 0-1.9%, 0-2%, 0-2.5%, 0-3%, 0-3.5%, 0- 4%, 0-4.5%, or 0-5%) HMW aggregates. In some embodiments, the eluate contains the anti-α4β7 antibody and contains about 2% or less (e.g., about 1.9% or less, 1.8% or less, 1.7% or less, 1.6% or less, 1.5% or less, 1.4% or less, 1.3% or less, 1.2% or less, 1.1% or less, 1% or less, 0.9% or less, 0.8% or less, 0.7% or less, 0.6% or less, 0.5% or less, 0.4% or less, 0.3% or less, 0.2% or less, or 0.1% or less) HMW aggregates. In particular embodiments, the Protein A resin eluate contains the anti-α4β7 antibody and contains about 0% to 2%, ≤2%, ≤1.9%, ≤1.8%, ≤1.7%, ≤1.6%, ≤1.5%, ≤1.4%, ≤1.3%, ≤1.2%, ≤1.1%, ≤1%, ≤0.9%, ≤0.8%, ≤0.7%, ≤0.6%, ≤0.5%, ≤0.4%, ≤0.3%, ≤0.2%, or ≤0.1% aggregates, e.g., HMW aggregates. In one embodiment, the eluate contains the anti-α4β7 antibody and contains about 1.2% or less HMW aggregates. In one embodiment, the eluate contains the anti-α4β7 antibody and contains about 1.1% or less HMW aggregates. In one embodiment, the eluate contains the anti-α4β7 antibody and contains about 1% or less (e.g., about 0.9% or less, 0.8% or less, 0.7% or less, 0.6% or less, 0.5% or less, 0.4% or less, 0.3% or less, 0.2% or less, or 0.1% or less) HMW aggregates. In one embodiment, the eluate contains the anti-α4β7 antibody and contains about 0.9% or less HMW aggregates.

The buffers and methods described herein can reduce the level of host cell protein (HCP) in a composition, such as a composition containing an anti-α4β7 antibody, eluted from a Protein A resin, relative to the level of HCP when an elution buffer is used that does not have one or more parameters described herein. In some embodiments, the Protein A resin eluate comprises a composition containing the anti-α4β7 antibody and less than about 250 ppm (e.g., less than about 240 ppm, 230 ppm, 220 ppm, 210 ppm, 200 ppm, 190 ppm, 180 ppm, 170 ppm, 160 ppm, 150 ppm, 140 ppm, 130 ppm, 120 ppm, 100 ppm, 90 ppm, 80 ppm, 70 ppm, 60 ppm, 50 ppm, 40 ppm, 30 ppm, 20 ppm, 10 ppm, 9 ppm, 8 ppm, 7 ppm, 6 ppm, 5 ppm, 4 ppm, 3 ppm, 2 ppm, or 1 ppm) HCP. In some embodiments, the Protein A resin eluate comprises a composition containing the anti-α4β7 antibody and about 1-250 ppm (e.g., about 1-240 ppm, 1-230 ppm, 1-220 ppm, 1-210 ppm, 1-200 ppm, 1-190 ppm, 1-180 ppm, 1-170 ppm, 1-160 ppm, 1-150 ppm, 1-140 ppm, 1-130 ppm, 1-120 ppm, 1-100 ppm, 1-90 ppm, 1-80 ppm, 1-70 ppm, 1-60 ppm, 1-50 ppm, 1-40 ppm, 1-30 ppm, 1-20 ppm, 1-10 ppm, 1-9 ppm, 1-8 ppm, 1-7 ppm, 1-6 ppm, 1-5 ppm, 1-4 ppm, 1-3 ppm, or 1-2 ppm) HCP.

In one embodiment, eluting an anti-α4β7 antibody bound to Protein A with an elution buffer having a pH of greater than 3.3 (e.g., pH 3.3-4.0, pH 3.4-4.0, pH 3.5-4.0, pH 3.6-4.0, pH 3.7-4.0, pH 3.8-4.0 or pH 3.9-4.0) results in an eluate comprising the anti-α4β7 antibody and a reduced level of HMW aggregate and/or an eluate comprising the anti-α4β7 antibody and a reduced level of HCP. In one embodiment, the elution solution has a pH of 3.3 to 3.9. In one embodiment, the elution buffer has a pH of 3.3 to 3.8. In an embodiment, the elution buffer pH is 3.4 to 3.6. In one embodiment, the elution buffer pH is 3.4-4.0. In one embodiment, the elution buffer comprises citric acid e.g., sodium citrate, e.g., 100 mM citric acid or 25 mM citric acid. In one embodiment, the Protein A affinity chromatography column is washed and eluted in a buffer comprising 25 mM sodium citrate, wherein the wash buffer is at a pH of 5.6 to 6.2, 5.7 to 5.9 or 5.8 and the elution buffer is at a pH of 3.3 to 3.9, 3.4 to 3.6 or 3.5.

In certain embodiments, the material loaded onto the Protein A resin is a clarified cell culture harvest, e.g., from a recombinant cell line expressing the anti-α4β7 antibody. In some embodiments, the recombinant cell line (i.e., host cell line) can be a Chinese Hamster Ovary (CHO) cell. In some embodiments, the CHO cell can be a GS-CHO cell, deficient in the gene encoding glutamine synthetase. In some embodiments, the CHO cell can be a DHFR-CHO cell, deficient in the gene encoding dihydrofolate reductase.

The Protein A eluate can be pH and/or conductivity adjusted for subsequent purification steps. The Protein A eluate may also be subjected to filtration through a depth filter to remove turbidity and/or various impurities from the antibody of interest prior to additional chromatographic polishing steps.

Purification of Anti-α4β7 Antibody Using a HIC Resin

An antibody, e.g., anti-α4β7 antibody (e.g., vedolizumab or an antibody having binding regions corresponding to vedolizumab), can also be purified using downstream process technologies following Protein A purification, as described in detail below, and as set forth in Example 2. Purification steps that are late in the downstream process are often referred to as “polishing” steps, and provide a unique challenge in that the level of impurity may be relatively low but even lower levels are desired given the nature an antibody intended for human use.

In one aspect, the methods described herein comprise the purification of an anti-α4β7 antibody from a liquid solution, e.g., a clarified harvest, comprising the antibody and one or more impurities using a hydrophobic interaction chromatography (HIC) resin.

In one embodiment, the present invention provides methods of reducing high molecular weight (HMW) aggregates from an anti-α4β7 antibody solution comprising contacting the antibody solution with a hydrophobic interaction chromatography (HIC) resin. Hydrophobic interaction chromatography (HIC) separates proteins according to differences in their surface hydrophobicity by utilizing a reversible interaction between these proteins and the hydrophobic surface of an HIC resin (e.g., polymeric matrix modified with hydrophobic ligands). Given the hydrophobic nature of an anti-α4β7 antibody like vedolizumab, a high hydrophobicity HIC resin can be used during the purification process to remove impurities, including HMW aggregates, residual protein A, and/or host cell proteins (HCP) contaminants where the an anti-α4β7 antibody flows through the HIC resin and does not bind. In some embodiments, a high hydrophobicity HIC resin suitable for use in the methods described herein comprises a polymethacrylate base material bonded with C6 groups, such as Toyopearl Hexyl-650C (Tosoh Biosciences).

In some embodiments, HIC is used in “flow-through mode.” Thus, “flow-through fractions,” as used herein, refers to protein in mobile phase buffer, collected in fractions, that has passed through a column containing resin, as provided herein.

In some embodiments, a solution comprising an anti-α4β7 antibody and at least one impurity is contacted with a hydrophobic interaction chromatography resin (HIC) resin under conditions that allow flow through of the anti-α4β7 antibody through the HIC resin. In one embodiment, the HIC resin has a mean pore size of about 100 nm and/or a pore size of about 100 μm. In one embodiment, the HIC resin is equilibrated with a buffer having a pH of less than about 7.2. In one embodiment, the HIC resin is equilibrated with a buffer having a pH of about 5.5 to about 7.2. In one embodiment, the HIC resin is equilibrated with a buffer having a pH of about 5.5 to about 7. In one embodiment, the buffer is a phosphate buffer. In one embodiment, the phosphate buffer comprises about 0.35 M to about 0.15 M potassium phosphate. In one embodiment, the resin load is about 55 to 75 mg/ml.

In one embodiment, a method of purifying an anti-α4β7 antibody with an HIC column comprises flowing the anti-α4β7 antibody-containing solution through a column, i.e., the purification comprises collecting the anti-α4β7 antibody in the column flow through and contaminants remain bound to the column, wherein the anti-α4β7 antibody and the column are in a solution comprising phosphate, e.g., potassium phosphate, at a concentration of 150 to 300 mM, 175 to 250 mM or about 200 mM at a pH of 5.2 to 6.5, 5.7 to 6.2 or about 5.9.

In some embodiments, such methods using a high hydrophobic HIC resin can be used to obtain a composition comprising the anti-α4β7 antibody and about 0% to 2.0% (e.g., less than 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or less than 2%) HMW aggregates. In some embodiments, such methods using a high hydrophobic HIC resin can be used to obtain a composition comprising the anti-α4β7 antibody and about 2% or less (e.g., about 1.9% or less, 1.8% or less, 1.7% or less, 1.6% or less, 1.5% or less, 1.4% or less, 1.3% or less, 1.2% or less, 1.1% or less, 1% or less, 0.9% or less, 0.8% or less, 0.7% or less, 0.6% or less, 0.5% or less, 0.4% or less, 0.3% or less, 0.2% or less, or 0.1% or less) HMW aggregates. In particular embodiments, such methods using a high hydrophobic HIC resin can be used to obtain a composition comprising the anti-α4β7 antibody and about 0% to 2%, ≤2%, ≤1.9%, ≤1.8%, ≤1.7%, ≤1.6%, ≤1.5%, ≤1.4%, ≤1.3%, ≤1.2%, ≤1.1%, ≤1%, ≤0.9%, ≤0.8%, ≤0.7%, ≤0.6%, ≤0.5%, ≤0.4%, ≤0.3%, ≤0.2%, or ≤0.1% HMW aggregates. In certain embodiments, such methods using a high hydrophobic HIC resin can be used to obtain a composition comprising the anti-α4β7 antibody and less than 0.6% HMW aggregate. In one embodiment, a composition comprising the anti-α4β7 antibody and less than 0.5% HMW aggregate is obtained. In one embodiment, a composition comprising the anti-α4β7 antibody and less than 0.4% HMW aggregate is obtained. Further, the composition may contain less than about 0.3 ppm host cell protein (HCP), wherein the host cell was a Chinese Hamster Ovary (CHO) cell, e.g., GS-CHO cell. In one embodiment, the composition comprises less than about 0.22 ppm residual protein A.

Purification of an Anti-α4β7 Antibody Using a Mixed Mode Chromatography Resin

In one aspect, provided herein are methods for the purification of an anti-α4β7 antibody, e.g., vedolizumab, from a liquid solution, e.g., a clarified cell culture harvest, comprising the antibody and one or more impurities, using mixed mode chromatography resin in bind/elute mode. In some embodiments, a mixed mode chromatography resin has properties suitable for high impurity removal and high capacity. In one embodiment, a mixed mode chromatography resin for purifying an anti-α4β7 antibody comprises strong anion exchange, hydrogen bonding and hydrophobic bonding capabilities. In another embodiment, a mixed mode chromatography resin for purifying an anti-α4β7 antibody comprises strong anion exchange, hydrogen bonding and hydrophobic bonding capabilities on a smaller bead, e.g., a bead of about 35-45 μm diameter. In certain embodiments, a mixed mode chromatography resin used in the methods and compositions described herein is CAPTO™ Adhere ImpRes (GE Healthcare Life Sciences, now Global Life Sciences Solutions, LLC). In certain embodiments, a mixed mode chromatography resin used in the methods and compositions described herein is CAPTO™ Adhere (GE Healthcare Life Sciences, now Global Life Sciences Solutions, LLC). The clarified cell culture harvest can be derived from host cells that recombinantly express the anti-α4β7 antibody. In certain embodiments, the host cell can be a Chinese Hamster Ovary (CHO) cell, such as a GS-CHO cell, or a DHFR-CHO cell.

The mixed mode chromatography methods provided herein include binding the anti-α4β7 antibody to a mixed mode chromatography resin. Additional purification steps, including but not limited to affinity chromatography (e.g., Protein A chromatography), anion exchange (AEX) chromatography, cation exchange (CEX) chromatography, and hydrophobic interaction chromatography (HIC) can be used before and/or after the mixed mode chromatography methods described herein. Accordingly, in some embodiments, the load material used for mixed mode chromatography may comprise a Protein A eluate, an AEX eluate, a CEX eluate, or a HIC eluate or collected HIC flow-through material. The mixed mode chromatography methods provided herein can further comprise, in some embodiments, washing the mixed mode resin with a wash solution, and eluting the antibody from the resin.

In certain embodiments, at least 25 g/L (e.g., at least 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, or 100 g/L) of antibody solution can be loaded onto the mixed mode chromatography resin. For example, at least 55 g/L of antibody solution can be loaded onto the mixed mode chromatography resin. In certain embodiments, about 25 g/L to about 100 g/L, such as about 25 g/L to about 95 g/L, about 25 g/L to about 90 g/L, about 25 g/L to about 85 g/L, about 25 g/L to about 80 g/L (e.g., about 30 g/L to about 80 g/L, about 35 g/L to about 80 g/L, about 40 g/L to about 80 g/L, about 45 g/L to about 80 g/L, about 50 g/L to about 80 g/L, about 55 g/L to about 80 g/L, about 60 g/L to about 80 g/L, about 65 g/L to about 80 g/L, about 70 g/L to about 80 g/L, or about 75 g/L to about 80 g/L) of antibody solution can be loaded onto the mixed mode chromatography resin. For example, about 55 g/L to about 80 g/L of antibody solution can be loaded onto the mixed mode chromatography resin.

The resin can optionally be washed with a suitable wash buffer, that will not elute the bound antibody from the resin. In one embodiment, the resin can optionally be washed with a sodium phosphate wash buffer. In some embodiments, the wash buffer can comprise 10 mM sodium phosphate, 25 mM sodium phosphate, 50 mM sodium phosphate, or 75 mM sodium phosphate at or around neutral pH (e.g., pH 6-8). Other suitable wash buffers compatible with mixed mode chromatography are widely available.

Described herein are buffers that increase the yield of an anti-α4β7 antibody and/or reduce the level of aggregates (e.g., % HMW species) in a preparation of an anti-α4β7 antibody following elution from a mixed mode chromatography resin. To increase the yield of an anti-α4β7 antibody and/or reduce the level of aggregates (e.g., % HMW species), the pH and/or conductivity of the mixed mode elution buffer can be modulated. Suitable elution solutions compatible with mixed mode chromatography are widely available. In some embodiments, a mixed mode chromatography elution solution comprises a buffer, such as citrate, acetate or phosphate.

In some embodiments, an elution buffer for use with a mixed mode chromatography resin in a method described herein has a pH at or above pH 3.5 (e.g., at or above pH 3.6, at or above pH 3.7, at or above pH 3.8, at or above pH 3.9, at or above pH 4.0, at or above pH 4.1, at or above pH 4.2, at or above pH 4.3, or at or above pH 4.4, or at or above pH 4.5). For example, an elution buffer for use with a mixed mode chromatography resin can have a pH at or above pH 3.9. In certain embodiments, an elution buffer for use with a mixed mode chromatography resin in a method described herein has a pH of about pH 3.9 to about pH 4.5 (e.g., a pH of about pH 3.9 to about pH 4.5, about pH 3.9 to about pH 4.4, about pH 3.9 to about pH 4.3, about pH 3.9 to about pH 4.2, about pH 3.9 to about pH 4.1, or about pH 3.9 to about pH 4.0). In some embodiments, an elution buffer for use with the mixed mode chromatography methods provided herein can have a pH of about pH 3.9 to about pH 4.4.

In additional or alternative embodiments, an elution buffer for use with a mixed mode chromatography resin in a method described herein has a pH at or below pH 4.5 (e.g., at or below pH 4.4, at or below pH 4.3, at or below pH 4.2, at or below pH 4.1, at or below pH 4.0, at or below pH 3.9, at or below pH 3.8, at or below pH 3.7, at or below pH 3.6, or at or below pH 3.5). For example, an elution buffer for use with a mixed mode chromatography resin can have a pH at or below pH 4.2. In certain embodiments, an elution buffer for use with a mixed mode chromatography resin in a method described herein has a pH of about pH 4.2 to about pH 3.5 (e.g., about pH 4.2 to about pH 3.6, about pH 4.2 to about pH 3.7, about pH 4.2 to about pH 3.8, about pH 4.2 to about pH 3.9, about pH 4.2 to about pH 4.0, or about pH 4.2 to about pH 4.1). For example, an elution buffer for use with a mixed mode chromatography resin can have a pH of about pH 4.2 to about pH 3.8.

In some embodiments, an elution buffer for use with a mixed mode chromatography resin in a method described herein has a conductivity of about 40 mS/cm or less (e.g., about 39 mS/cm, 38 mS/cm, 37 mS/cm, 36 mS/cm, 35 mS/cm, 34 mS/cm, 33 mS/cm, 32 mS/cm, 31 mS/cm, 30 mS/cm, 29 mS/cm, 28 mS/cm, 27 mS/cm, 26 mS/cm, 25 mS/cm, 24 mS/cm, 23 mS/cm, 22 mS/cm, 21 mS/cm, 20 mS/cm, 19 mS/cm, 18 mS/cm, 17 mS/cm, 16 mS/cm, 15 mS/cm, 14 mS/cm, 13 mS/cm, 12 mS/cm, 11 mS/cm, or 10 mS/cm or less). For example, an elution buffer for use with a mixed mode chromatography resin can have a conductivity of about 30 mS/cm or less. In certain embodiments, an elution buffer for use with a mixed mode chromatography resin in a method described herein has a conductivity of about 10 mS/cm to about 40 mS/cm, such as about 15 mS/cm to about 35 mS/cm or about 20 mS/cm to about 30 mS/cm. For example, an elution buffer for use with a mixed mode chromatography resin can have a conductivity of about 20 mS/cm to about 30 mS/cm.

In additional or alternative embodiments, an elution buffer for use with a mixed mode chromatography resin in a method described herein has a conductivity at or below 30 mS/cm (e.g., at or below 29 mS/cm, 28 mS/cm, 27 mS/cm, 26 mS/cm, 25 mS/cm, 24 mS/cm, 23 mS/cm, 22 mS/cm, 21 mS/cm, 20 mS/cm, 19 mS/cm, 18 mS/cm, 17 mS/cm, 16 mS/cm, 15 mS/cm, 14 mS/cm, 13 mS/cm, 12 mS/cm, 11 mS/cm, or 10 mS/cm). For example, an elution buffer for use with a mixed mode chromatography resin can have a conductivity at or below 28 mS/cm. In certain embodiments, an elution buffer for use with a mixed mode chromatography resin in a method described herein has a conductivity of about 10 mS/cm to about 40 mS/cm (e.g., about 15 mS/cm to about 35 mS/cm, about 18 mS/cm to about 35 mS/cm, about 11 mS/cm to about 30 mS/cm, about 12 mS/cm to about 30 mS/cm, about 13 mS/cm to about 30 mS/cm, about 14 mS/cm to about 30 mS/cm, about 15 mS/cm to about 30 mS/cm, about 16 mS/cm to about 30 mS/cm, about 17 mS/cm to about 30 mS/cm, about 18 mS/cm to about 30 mS/cm, about 19 mS/cm to about 30 mS/cm, about 20 mS/cm to about 30 mS/cm, about 21 mS/cm to about 30 mS/cm, about 22 mS/cm to about 30 mS/cm, about 23 mS/cm to about 30 mS/cm, about 24 mS/cm to about 30 mS/cm, about 25 mS/cm to about 30 mS/cm, about 26 mS/cm to about 30 mS/cm, or about 27 mS/cm to about 30 mS/cm). For example, in some embodiments, an elution buffer for use with a mixed mode chromatography resin can have a conductivity of about 18 mS/cm to about 28 mS/cm.

In some embodiments, an elution buffer for use with a mixed mode chromatography resin in a method described herein can comprise an ionic salt, e.g., NaCl, at a concentration of about 100-300 mM (e.g., about 110-290 mM, 120-280 mM, 130-270 mM, 140-260 mM, 150-250 mM, 160-240 mM, 170-230 mM, 180-220 mM, or 190-210 mM). For example, an elution buffer for use with a mixed mode chromatography resin can have NaCl at a concentration of about 160 mM to about 240 mM. In certain embodiments, an elution buffer for use with a mixed mode chromatography resin in a method described herein has NaCl at a concentration of about 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or 300 mM.

In some embodiments, a method for the purification of an anti-α4β7 antibody, e.g., vedolizumab, from a liquid solution, e.g., a clarified cell culture harvest, using mixed mode chromatography resin comprises loading the anti-α4β7 antibody onto the column comprising the mixed mode resin at a concentration of 40 to 90, 50 to 80 or about 65 g protein/L resin, washing the column and eluting the column with an elution buffer, e.g., a sodium citrate buffer, at a pH of 3.5 to 4.5, 3.9 to 4.4 or about 4.1. In some embodiments, the method further comprises including an ionic salt, e.g., NaCl, so the conductivity of the elution buffer is 15 to 35, 20 to 30 or about 24 mS/cm. In some embodiments, the method comprises loading the antibody onto the column comprising the mixed mode resin at a concentration of 53-77 g protein/L resin. In some embodiments, the method comprises eluting the antibody from the column using an elution buffer having a pH of about 3.9-4.4, and a conductivity of about 20-28 mS/cm.

In some embodiments, purification of an anti-α4β7 antibody can be achieved using a mixed mode chromatography method described herein in conjunction with cation exchange (CEX) chromatography.

In some embodiments, the methods described herein can improve the yield of an anti-α4β7 antibody eluted from a mixed mode chromatography column, relative to the yield of a suitable control process where an elution buffer is used that does not have one or more parameters described herein, e.g., relative to a process performed using an elution buffer having a pH of 3.7 or less, 3.6 or less, 3.5 or less, 3.3 or less, or 3.0 or less. In some embodiments, the yield is increased by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more. In some embodiments, the buffers and methods described herein can result in 50% or more (e.g., 55%, 60%, 65%,70%, 75%, 80%, 85%, 90%, 95%, 96%, 97%, 98%, 99%, or more) recovery of an anti-α4β7 antibody eluted from a mixed mode chromatography column. In certain embodiments, the buffers and methods described herein can result in 50-95% (e.g., 55-95%, 60-95%, 65-95%, 70-95%, 75-95%, 80-95%, 85-95%, 90-95%, or more) recovery of an anti-α4β7 antibody eluted from a mixed mode chromatography column.

In some embodiments, such compositions and methods using a mixed mode chromatography resin can be used to obtain a composition comprising an anti-α4β7 antibody and about 0% to 2.0% (e.g., 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2%) HMW aggregates. In some embodiments, such methods using a mixed mode chromatography resin can be used to obtain a composition comprising an anti-α4β7 antibody and about 2% or less (e.g., about 1.9% or less, 1.8% or less, 1.7% or less, 1.6% or less, 1.5% or less, 1.4% or less, 1.3% or less, 1.2% or less, 1.1% or less, 1% or less, 0.9% or less, 0.8% or less, 0.7% or less, 0.6% or less, 0.5% or less, 0.4% or less, 0.3% or less, 0.2% or less, or 0.1% or less) HMW aggregates. In particular embodiments, such methods using a mixed mode chromatography resin can be used to obtain a composition comprising an anti-α4β7 antibody and about 0% to 2%, ≤2%, ≤1.9%, ≤1.8%, ≤1.7%, ≤1.6%, ≤1.5%, ≤1.4%, ≤1.3%, ≤1.2%, ≤1.1%, ≤1%, ≤0.9%, ≤0.8%, ≤0.7%, ≤0.6%, ≤0.5%, ≤0.4%, ≤0.3%, ≤0.2%, or ≤0.1% aggregates. In other embodiments, the level of HMW aggregates is reduced by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more, relative to level of HMW aggregates in the load material. In some embodiments, the mixed mode chromatography methods provided herein can be used to reduce the level of HMW aggregates in a composition comprising an anti-α4β7 antibody, relative to the level of HMW aggregates obtained from a suitable control process where an elution buffer is used that does not have one or more parameters described herein, e.g., relative to a process performed using an elution buffer having a pH of 3.7 or less, 3.6 or less, 3.5 or less, 3.3 or less, or 3.0 or less. In some embodiments, the level of HMW aggregates is reduced by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more, relative to a suitable control.

Purification of an Anti-α4β7 Antibody Using a Cation Exchange (CEX) Resin

In one aspect, provided herein are methods for the purification of an anti-α4β7 antibody, e.g., vedolizumab, from a liquid solution, e.g., a clarified cell culture harvest, comprising the antibody and one or more impurities, using a cation exchange (CEX) resin in bind/elute mode. In some embodiments, a CEX resin for the purification of an anti-α4β7 antibody is a strong cation exchange resin. In certain embodiments, a CEX resin compatible for use in the methods and compositions described herein comprises an —SO3⁻ functional group. For example, in some embodiments, the CEX resin is Nuvia HR-S. The clarified cell culture harvest can be derived from host cells that recombinantly express the anti-α4β7 antibody. In certain embodiments, the host cell can be a Chinese Hamster Ovary (CHO) cell, such as a GS-CHO cell, or a DHFR-CHO cell.

The CEX methods provided herein include binding the anti-α4β7 antibody to a cation exchange chromatography resin. Additional purification steps, including but not limited to affinity chromatography (e.g., Protein A chromatography), anion exchange (AEX) chromatography, mixed mode chromatography, and hydrophobic interaction chromatography (HIC) can be used before and/or after the CEX methods described herein. Accordingly, in some embodiments, the load material used for CEX chromatography may comprise a Protein A eluate, an AEX eluate, a mixed mode eluate, or a HIC eluate. The CEX methods provided herein can further comprise, in some embodiments, washing the CEX resin with a wash solution, and eluting the antibody from the resin. Suitable solutions, e.g., for loading, washing and eluting a protein, e.g., an anti-α4β7 antibody, compatible with CEX chromatography are widely available. In some embodiments, CEX chromatography solutions comprise a buffer, such as citrate, acetate or phosphate.

In certain embodiments, at least 20 g/L (e.g., at least 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, or 100 g/L) of antibody solution can be loaded onto the CEX resin. For example, at least 25 g/L of antibody solution can be loaded onto the CEX resin. In certain embodiments, about 25-100 g/L (e.g., about 25-90 g/L, 25-80 g/L, 25-70 g/L, 25-60 g/L, 25-50 g/L, 25-40 g/L, or 25-30 g/L) of antibody solution can be loaded onto the CEX resin. In certain embodiments, about 25-70 g/L (e.g., about 25-65 g/L, 30-60 g/L, 35-55 g/L, or 40-50 g/L) of antibody solution can be loaded onto the CEX resin. For example, about 30-60 g/L of antibody solution can be loaded onto the CEX resin.

The resin can optionally be washed with a suitable wash buffer, that will not elute the bound antibody from the resin. In some embodiments, the wash buffer has the same composition as the buffer used to load the antibody onto the resin. In one embodiment, the resin can optionally be washed with a sodium acetate buffer, e.g., 25 mM sodium acetate, 50 mM sodium acetate, 75 mM sodium acetate, or 100 mM sodium acetate. In some embodiments, the wash buffer has a pH in the range of pH 5-7, e.g., pH 5-6, pH 5.5-6.5, pH 5.1-5.8, pH 5.3-5.6, pH 6-7 or pH 5.4. Other suitable wash buffers compatible with CEX chromatography are widely available.

The pH and/or conductivity of the elution buffer can be adjusted to modulate the level of HMW aggregates, the level of major (main) isoform species, the level of acidic isoform species, and/or the level of basic isoform species in the preparation of anti-α4β7 antibody eluted from the CEX resin. In some embodiments, an elution buffer for use with a CEX resin in a method described herein has a pH at or below pH 6.0 (e.g., at or below pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7, pH 5.8, pH 5.9, or pH 6.0). In certain embodiments, an elution buffer for use with a CEX resin in a method described herein has a pH of about pH 4.5 to about pH 6.0 (e.g., a pH of about pH 4.5 to about pH 5.8, about pH 4.9 to about pH 5.9, about pH 5.0 to about pH 6.0, about pH 5.0 to about pH 5.9, about pH 5.0 to about pH 5.8, about pH 5.0 to about pH 5.7, about pH 5.0 to about pH 5.6, or about pH 5.0 to about pH 5.5). For example, an elution buffer for use with a CEX resin can have a pH of about pH 5.1 to about pH 5.8. In some embodiments, the pH of the CEX elution buffer is the same as the wash buffer.

In additional or alternative embodiments, an elution buffer for use with a CEX resin in a method described herein has a conductivity at or below 20 mS/cm (e.g., at or below 19 mS/cm, 18 mS/cm, 17 mS/cm, 16 mS/cm, 15 mS/cm, 14 mS/cm, 13 mS/cm, 12 mS/cm, 11 mS/cm, or 10 mS/cm). For example, an elution buffer for use with a CEX resin can have a conductivity at or below 16 mS/cm. In some embodiments, an elution buffer for use with a CEX resin in a method described herein has a conductivity of about 10 mS/cm to about 20 mS/cm (e.g., about 10 mS/cm to about 19 mS/cm, about 10 mS/cm to about 18 mS/cm, about 10 mS/cm to about 17 mS/cm, about 10 mS/cm to about 16 mS/cm, about 10 mS/cm to about 15 mS/cm, about 10 mS/cm to about 14 mS/cm, about 10 mS/cm to about 13 mS/cm, or about 10 mS/cm to about 12 mS/cm). In some embodiments, an elution buffer for use with a CEX resin can have a conductivity of about 11 mS/cm to about 16 mS/cm. Additionally, or alternatively, an elution buffer for use with a CEX resin can have a conductivity at or below 14 mS/cm. In certain embodiments, an elution buffer for use with a CEX resin in a method described herein has a conductivity of about 11 mS/cm to about 14 mS/cm, such as about 12 mS/cm to about 14 mS/cm or about 13 mS/cm to about 14 mS/cm. For example, an elution buffer for use with a CEX resin can have a conductivity of about 12 mS/cm to about 14 mS/cm. Additionally, or alternatively, an elution buffer for use with a CEX resin can have a conductivity at or above 11 mS/cm (e.g., at or above 12 mS/cm, 13 mS/cm, 14 mS/cm, 15 mS/cm, 16 mS/cm, 17 mS/cm, 18 mS/cm, 19 mS/cm, or 20 mS/cm). For example, an elution buffer for use with a CEX resin can, in some embodiments, have a conductivity at or above 12 mS/cm.

In some embodiments, an elution buffer for use with a CEX resin in a method described herein can have NaCl at a concentration of about 50 mM, 60 mM, 70 mM, 80 mM, 90 mM, 100 mM, 110 mM, 120 mM, 130 mM, 140 mM, or 150 mM. For example, an elution buffer for use with a CEX resin can have NaCl at a concentration of about 90-120 mM. In certain embodiments, an elution buffer for use with a CEX resin in a method described herein has NaCl at a concentration of about 50-150 mM (e.g., about 50-140 mM, 60-130 mM, 70-120 mM, 80-110 mM, or 90-100 mM). In particular embodiments, an elution buffer for use with a CEX resin in a method described herein has NaCl at a concentration of about 70-120 mM (e.g., about 70-110 mM, 70-100 mM, 70-90 mM, or 70-80 mM. For example, an elution buffer for use with a CEX resin can have NaCl at a concentration of about 70-110 mM.

In some embodiments, a method for the purification of an anti-α4β7 antibody, e.g., vedolizumab, from a liquid solution, e.g., a clarified cell culture harvest, using a CEX resin, e.g., a strong cation exchange resin, comprises loading the anti-α4β7 antibody onto the column comprising the CEX resin at a concentration of 40 to 90 , 50 to 65 or about 57 g protein/L resin, washing the column and eluting the column with a buffer, e.g., a sodium acetate buffer, at a pH of 5 to 6, 5.2 to 5.6 or about 5.4. In some embodiments, the method further comprises including an ionic salt, e.g., NaCl, so the conductivity of the elution buffer is 5 to 25, 10 to 17 or about 13 mS/cm. In other embodiments, a method for the purification of an anti-α4β7 antibody, e.g., vedolizumab, from a liquid solution, e.g., a clarified cell culture harvest, using a CEX resin, e.g., a strong cation exchange resin, comprises eluting the column with a buffer, e.g., a sodium acetate buffer, having a pH of 5 to 6, 5.2 to 5.6 or about 5.4, and a conductivity of 5 to 25, 10 to 15 or about 13 mS/cm. In one embodiment, the method comprises eluting the column with an elution buffer having a pH of about 5.4, and a conductivity of about 13 mS/cm. In some embodiments, the CEX resin is loaded with the antibody at about 57 g protein/L resin.

In some embodiments, purification of an anti-α4β7 antibody can be achieved using a CEX resin as described herein in conjunction with mixed mode chromatography.

In some embodiments, the CEX methods described herein can be used to obtain a composition comprising an anti-α4β7 antibody and about 0% to 2.0% (e.g., about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2%) HMW aggregates. In some embodiments, such methods using a CEX resin can be used to obtain a composition comprising an anti-α4β7 antibody and about 2% or less (e.g., about 1.9% or less, 1.8% or less, 1.7% or less, 1.6% or less, 1.5% or less, 1.4% or less, 1.3% or less, 1.2% or less, 1.1% or less, 1% or less, 0.9% or less, 0.8% or less, 0.7% or less, 0.6% or less, 0.5% or less, 0.4% or less, 0.3% or less, 0.2% or less, 0.1% or less, 0.09% or less, 0.08% or less, 0.07% or less, 0.06% or less, 0.05% or less, 0.04% or less, 0.03% or less, 0.02% or less, or 0.01% or less) HMW aggregates. In particular embodiments, such methods using a CEX resin can be used to obtain a composition comprising an anti-α4β7 antibody and about 0% to 2%, ≤2%, ≤1.9%, ≤1.8%, ≤1.7%, ≤1.6%, ≤1.5%, ≤1.4%, ≤1.3%, ≤1.2%, ≤1.1%, ≤1%, ≤0.9%, ≤0.8%, ≤0.7%, ≤0.6%, ≤0.5%, ≤0.4%, ≤0.3%, ≤0.2%, ≤0.1%, ≤0.09%, ≤0.08%, ≤0.07%, ≤0.06%, ≤0.05%, ≤0.04%, ≤0.03%, ≤0.02%, or ≤0.01% aggregates, e.g., HMW aggregates. In other embodiments, the level of HMW aggregates is reduced by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more, relative to level of HMW aggregates in the load material. In some embodiments, the CEX methods provided herein can be used to reduce the level of HMW aggregates in a composition comprising an anti-α4β7 antibody, relative to the level of HMW aggregates in a preparation of the anti-α4β7 antibody obtained using a suitable control CEX process where an elution buffer is used that does not have one or more parameters described herein, e.g., relative to a process performed using an elution buffer having a pH of 6.3 or more, 6.5 or more, 6.7 or more, or 6.9 or more, and/or a conductivity of 18 mS/cm or more, 19 mS/cm or more, 20 mS/cm or more, 22 mS/cm or more, or 24 mS/cm or more. In some embodiments, the level of HMW aggregates is reduced by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more, relative to a suitable control.

In some embodiments of the methods provided herein, the pH and/or conductivity of the elution buffer used to elute the anti-α4β7 antibody from the CEX resin can be used modulate the isoform distribution of the anti-α4β7 antibody present in the eluate. For example, the pH and/or conductivity of the elution buffer can be used to increase the percentage of the major (main) antibody isoform, reduce the percentage of acidic isoform species, and/or reduce the percentage of basic isoform species.

In some embodiments, an elution buffer can be selected having a pH of 6.0 or less, e.g., 5.9 or less, 5.8 or less, 5.7 or less, 5.6 or less, 5.5 or less, 5.4 or less, 5.3 or less, or 5.2 or less, e.g., pH 4.5-6.0, pH 4.5-5.5, or pH 5.0-6.0. In some embodiments, an elution buffer can be selected having a conductivity of at least 10 mS/cm, e.g., at least 11 mS/cm, at least 12 mS/cm, at least 13 mS/cm, at least 14 mS/cm, at least 15 mS/cm, at least 16 mS/cm or more, e.g., 10-17 mS/cm, 12-17 mS/cm, 13-17 mS/cm, 14-17 mS/cm, 15-17 mS/cm, 16-17 mS/cm, 10-16 mS/cm, 12-16 mS/cm, 13-16 mS/cm, 14-16 mS/cm, or 15-16 mS/cm. In some embodiments, the foregoing elution buffer conditions can be used to obtain a composition containing at least 60%, 61%, 62%, 63%, 64%, 65%, 66%, 67%, 68%, 69%, 70%, 71%, 72%, 73%, 74%, 75%, or more major isoform of an anti-α4β7 antibody. In some embodiments, the foregoing elution buffer conditions can be used to obtain a composition that contains 20% or less (e.g., about 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, or 1% or less) basic isoform species. In particular embodiments, such methods using a CEX resin can be used to obtain a composition comprising a major isoform of an anti-α4β7 antibody and about ≤20%, ≤19%, ≤18%, ≤17%, ≤16%, ≤15%, ≤14%, ≤13%, ≤12%, ≤11%, ≤10%, ≤9%, ≤8%, ≤7%, ≤6%, ≤5%, ≤4%, ≤3%, ≤2%, or ≤1% basic isoform species. In other embodiments, the level of basic isoform species is reduced by at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 96%, 97%, 98%, 99%, or more, relative to level of basic isoforms species in the load material.

### III. Analytical Methods

In certain embodiments, the levels of aggregates, monomer, and fragments in the chromatographic samples produced using the techniques described herein are analyzed. In certain embodiments, the aggregates, monomer, and fragments are measured using a size exclusion chromatographic (SEC) method for each molecule. For example, but not by way of limitation, a TSK-gel G3000SWxL, 5 μm, 125 Å, 7.8×300 mm column (Tosoh Bioscience) can be used in connection with certain embodiments, while a TSK-gel Super SW3000, 4 μm, 250 Å, 4.6×300 mm column (Tosoh Bioscience) can be used in alternative embodiments. In certain embodiments, the aforementioned columns are used along with an Agilent or a Shimazhu HPLC system. In certain embodiments, sample injections are made under isocratic elution conditions using a mobile phase consisting of, for example, 100 mM sodium sulfate and 100 mM sodium phosphate at pH 6.8, and detected with UV absorbance at 214 nm. In certain embodiments, the mobile phase will consist of 1×PBS at pH 7.4, and elution profile detected with UV absorbance at 280 nm. In certain embodiments, quantification is based on the relative area of detected peaks.

Any additional technique, such as mass spectroscopy, can be used for assaying size variants.

Various parameters of an antibody, or antigen binding portion thereof, reported herein can be measured using standard analytical methods and techniques, such as those described below.

In various embodiments set forth herein, cation exchange chromatography (CEX) can be used to determine the relative amounts of the major isoform, basic isoform(s), and acidic isoform(s) present in a population of an antibody or antigen binding portion thereof, e.g., vedolizumab. The CEX method fractionates antibody species according to overall surface charge. After dilution to low ionic strength using mobile phase, the test sample can be injected onto a CEX column, such as for example a Dionex Pro-Pac™ WCX-10 column (Thermo Fisher Scientific, Waltham, Mass. (USA)), equilibrated in a suitable buffer, e.g., 10 mM sodium phosphate, pH 6.6. The antibody can be eluted using a sodium chloride gradient in the same buffer. Protein elution can be monitored at 280 nm, and peaks are assigned to acidic, basic, or major isoforms categories. Acidic peaks elute from the column with a shorter retention time than the major isoform peak, and basic peaks elute from the column with a longer retention time than the major isoform peak. The percent major isoform, the sum of percent acidic species, and the sum of percent basic species are reported. The major isoform retention time of the sample is compared with that of a reference standard to determine the conformance. In one embodiment, a CEX assay method comprises diluting a test sample to low ionic strength, injecting onto a CEX column which is equilibrated in 10 mM sodium phosphate, pH 6.6, eluting the column with a NaCl gradient in this buffer, monitoring the peaks at 280 nm and assigning peaks as acidic, main or basic, wherein the acidic peaks elute first with the shortest retention times, the main peak elutes second and the basic peaks elute with the longest retention times, and the peak areas are quantified and their amounts are calculated as the percent of all the peak area.

In various embodiments set forth herein, size exclusion chromatography (SEC) can be used to determine the relative level of monomers, high molecular weight (HMW) aggregates, and low molecular weight (LMW) degradation products present in a population of an antibody or antigen binding portion thereof, e.g., vedolizumab. The SEC method provides size-based separation of antibody monomer from HMW species and LMW degradation products. Test samples and reference standards can be analyzed using commercially available SEC columns, using an appropriate buffer. For example, in some embodiments, SEC analysis can be performed using a G3000 SWxl column (Tosoh Bioscience, King of Prussia, Pa. (USA)), or two G3000 SWxl columns connected in tandem, and an isocratic phosphate-sodium chloride buffer system, pH 6.8. Elution of protein species is monitored at 280 nm. The main peak (monomer) and the total peak area are assessed to determine purity. In one embodiment, the SEC analysis comprises injecting a sample onto two G3000 SWxl columns connected in tandem, and run in an isocratic phosphate-sodium chloride buffer system, pH 6.8, wherein the elution of protein species is monitored at 280 nm and the main peak (monomer) and the total peak area are measured. The purity (%) of the sample (calculated as % monomer), the % HMW aggregate, and/or the % LMW degradation product are reported.

Residual CHO host cell protein (HCP) impurities present in an antibody preparation can be measured if desired by enzyme-linked immunosorbent assay (ELISA), using standard techniques. Many ELISA kits designed for this purpose are commercially available, such as the CHO HCP ELISA Kit 3G from Cygnus Technologies (Southport, N.C. (USA)). Host cell proteins in a test sample can be captured using an immobilized polyclonal anti-CHO HCP antibody. Captured proteins can then be detected using a suitable detection agent, for example, a horseradish peroxidase-labeled version of the same antibody. In this exemplary embodiment, the amount of captured peroxidase, which is directly proportional to the concentration of CHO HCP, can be measured colorimetrically at 450 nm using the peroxidase substrate 3,3′,5,5′-tetramethylbenzidine (TMB). Accordingly, the CHO HCP assay comprises using a polyclonal anti-CHO HCP antibody to capture HCP, which is detected after binding a horseradish peroxidase-labeled version of the polyclonal anti-CHO HCP antibody which converts the peroxidase substrate 3,3′,5,5′-tetramethylbenzidine (TMB) to a substance that is quantified colorimetrically at 450 nm. The HCP concentration can be determined by comparison to a CHO HCP standard curve, such as that included in the test kit, and is reported as a percentage of the total level of protein in the antibody preparation.

### IV. Downstream Processing and Formulation

The anti-α4β7 antibody (e.g., vedolizumab or an antibody having binding regions corresponding to vedolizumab) can be further purified from contaminant soluble proteins and polypeptides, with the following procedures being exemplary of suitable purification procedures, that can optionally be used alone or in combination, in conjunction with one or more of the methods provided herein: affinity chromatography, e.g. using a resin that binds an Fc region of an antibody, such as Protein A; fractionation on an ion-exchange column or resin such as cation exchange chromatography (CEX), e.g., SP-Sepharose™ or CM-Sepharose™ hydroxyapatite; anion exchange chromatography (AEX); hydrophobic interaction chromatography (HIC); mixed mode chromatography; ethanol precipitation; chromatofocusing; ammonium sulfate precipitation; gel filtration using, for example, Sephadex G-75™; ultrafiltration and/or diafiltration, or combinations of the foregoing. Examples of purification methods are described in Liu et al., mAbs, 2:480-499 (2010). At the end of the purification process, the recombinant protein is highly pure and is suitable for human therapeutic use, e.g., in pharmaceutical antibody formulations described below. Following purification, the highly pure recombinant protein may be ultrafilered/diafiltered (UF/DF) into a pharmaceutical formulation suitable for human administration.

Following diafiltration and ultrafiltration, the antibody formulation may remain as a liquid or be lyophilized into a dry antibody formulation. In one aspect, the dry, lyophilized antibody formulation is provided in a single dose vial comprising 180 mg, 240 mg, 300 mg, 360 mg, 450 mg or 600 mg of anti-α4β7 antibody and can be reconstituted with a liquid, such as sterile water, for administration. In another aspect, the anti-α4β7 antibody, e.g., vedolizumab, is in a stable liquid pharmaceutical composition stored in a container, e.g., a vial, a syringe or cartridge, at about 2-8° C. until it is administered to a subject in need thereof. In some embodiments, the reconstituted lyophilized formulation or the stable liquid pharmaceutical composition of anti-α4β7 antibody comprises about 0% to 5.0%, 0% to 2%, ≤2%, ≤1%, ≤0.6% or ≤0.5% aggregates.

Accordingly, in some embodiments, provided herein is a reconstituted lyophilized antibody formulation or a stable liquid pharmaceutical composition comprising a humanized anti-α4β7 antibody, or antigen binding portion thereof. Examples of lyophilized formulations comprising an anti-α4β7 antibody, e.g., vedolizumab, are described in U.S. Pat. No. 9,764,033, the contents of which are incorporated herein by reference. Examples of liquid formulations comprising an anti-α4β7 antibody, e.g., vedolizumab, are described in U.S. Pat. No. 10,040,855, the contents of which are incorporated herein by reference. In some embodiments, the reconstituted lyophilized formulation or the stable liquid pharmaceutical composition of anti-α4β7 antibody comprises about 11% to 16%, 12% to 15%, ≤14%, ≤13%, ≤12%, or ≤11% basic isoform species. In some embodiments, the reconstituted lyophilized formulation or the stable liquid pharmaceutical composition of anti-α4β7 antibody comprises 65% to 75%, 66% to 74%, 67% to 73%, at least 65%, at least 66%, at least 67%, at least 68%, at least 69%, or at least 70% major isoform.

Purified antibody, e.g., anti-α4β7 antibody (e.g., vedolizumab or an antibody having binding regions corresponding to vedolizumab) may be concentrated to provide a concentrated protein composition, e.g., one with an antibody concentration of at least 100 mg/mL or 125 mg/mL or 150 mg/mL or a concentration of about 100 mg/mL or 125 mg/mL or 150 mg/mL. It is understood that concentrated antibody product may be concentrated up to levels that are permissible under the concentration conditions, e.g., up to a concentration at which the polypeptide is no longer soluble in solution.

In some embodiments, the compositions obtained herein comprise purified anti-α4β7 antibody, such as vedolizumab, and are subsequently formulated for human use. In one embodiment, purified antibody is formulated into a dry, lyophilized formulation which can be reconstituted with a liquid, such as sterile water, for administration. Administration of a reconstituted formulation can be by parenteral injection by one of the routes described above. An intravenous injection can be by infusion, such as by further dilution with sterile isotonic saline, buffer, e.g., phosphate-buffered saline or Ringer's (lactated or dextrose) solution. In some embodiments, purified antibody is formulated into a liquid formulation so that the anti-α4β7 antibody is administered by subcutaneous injection, e.g., a dose of about 54 mg, 108 mg or about 165 mg or about 216 mg.

Containers that can be used to store and freeze purified compositions described herein include polycarbonate bottles (for IV formulations) or PETG bottles (for subcutaneous formulations). Following aliquoting the formulations to a bottle, freezing may occur (e.g., at −60 degrees Celsius or less).

The following examples exemplify improved methods and compositions for purification of antibodies. Examples 1 to 5 below describe various methods and compositions that may be used to obtain purified compositions of an anti-α4β7 antibody, particularly vedolizumab. Included herein are methods described in the Examples below, including the various parameters described therein.

### EXAMPLE

The below examples describe the purification process of vedolizumab which was produced in a cell culture using CHO cells as an expression system.

### Example 1: Effect of Elution Buffer on Purification of Vedolizumab Using a Protein A Resin

This Example demonstrates antibody purification methods using Protein A resin, which can be used in the production of a therapeutic anti-α4β7 antibody, e.g., vedolizumab. As described herein, modulation of elution pH off of a Protein A resin resulted in a reduced level of aggregates in the purified composition of vedolizumab.

Vedolizumab was produced by cell culture of recombinant Chinese Hamster Ovary (CHO) cells (GS-CHO) genetically engineered to express the antibody (for general cell culture methods see, Li et al. (2010) mAbs 2:5, 466-477).

Following cell culture in CHO cells, selective capture of vedolizumab was carried out following primary recovery using a Protein A affinity column. Affinity chromatography was carried out using a recombinant Protein A resin to selectively remove the antibody from the clarified harvest derived from the upstream primary recovery process. The step also removed process-related impurities such as host cell proteins (HCPs).

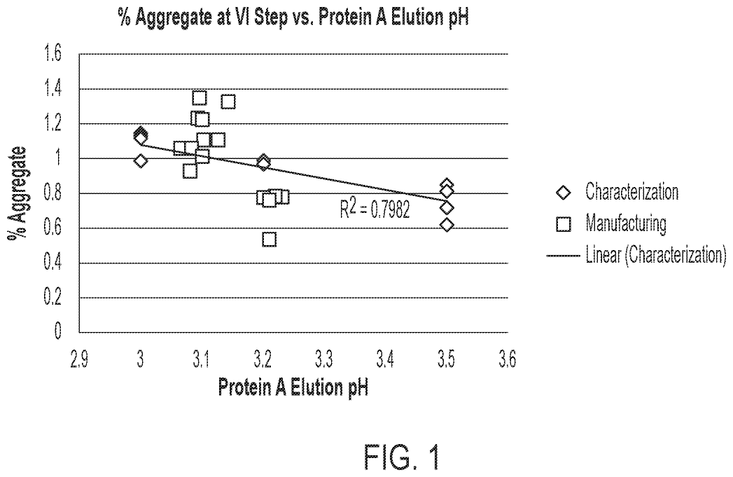

The Protein A resin was first equilibrated with a PBS equilibration solution (pH 7.2). The clarified harvest was then loaded. Three washes were carried out. Wash 1 was done with a PBS wash solution (pH 7.2) identical to the equilibration solution; Wash 2 was done with a 1 M NaCl, PBS wash solution (pH 7.2); and Wash 3 was done with the same wash solution as Wash 1 (PBS) and the solution used for equilibration. The washes served to wash impurities from the antibody as it remained bound to the resin. The antibody was then eluted from the resin using elution buffers with a range of pH. As described in FIG. 1, elution buffers having a pH of 3 to 3.5 were tested. The results provided in FIG. 1 show that with increasing pH, the % of aggregate decreased. As described in FIG. 1, eluting vedolizumab from Protein A at a pH of 3 resulted in an eluate having a higher level of aggregate, i.e., about 1-1.2%, whereas an eluate obtained from eluting the antibody using an elution buffer having a higher pH, e.g., a pH of about 3.5, had about 0.6-0.85% aggregate.

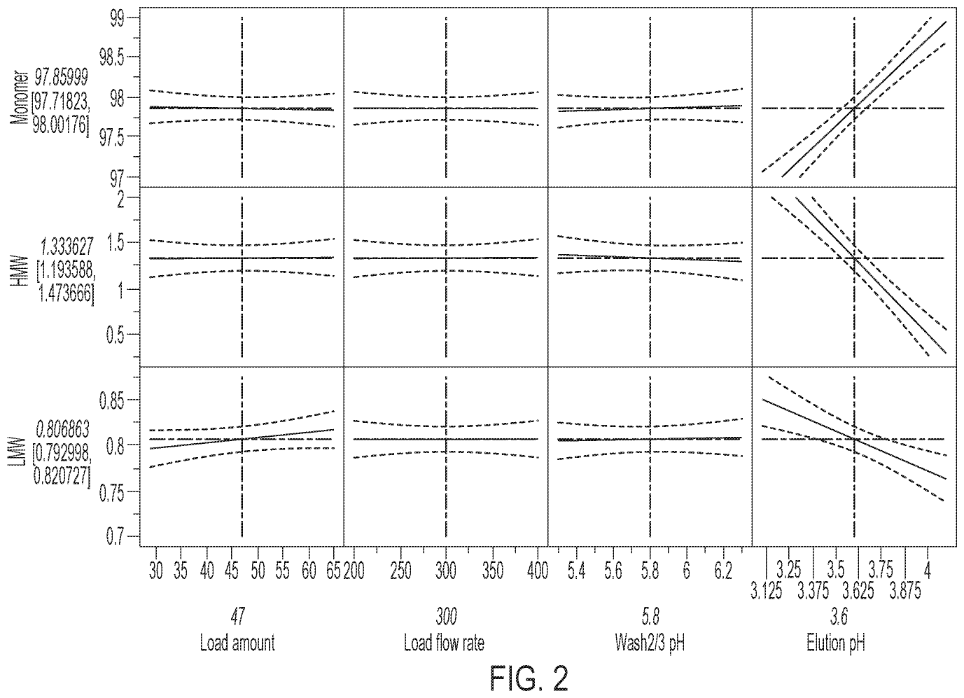

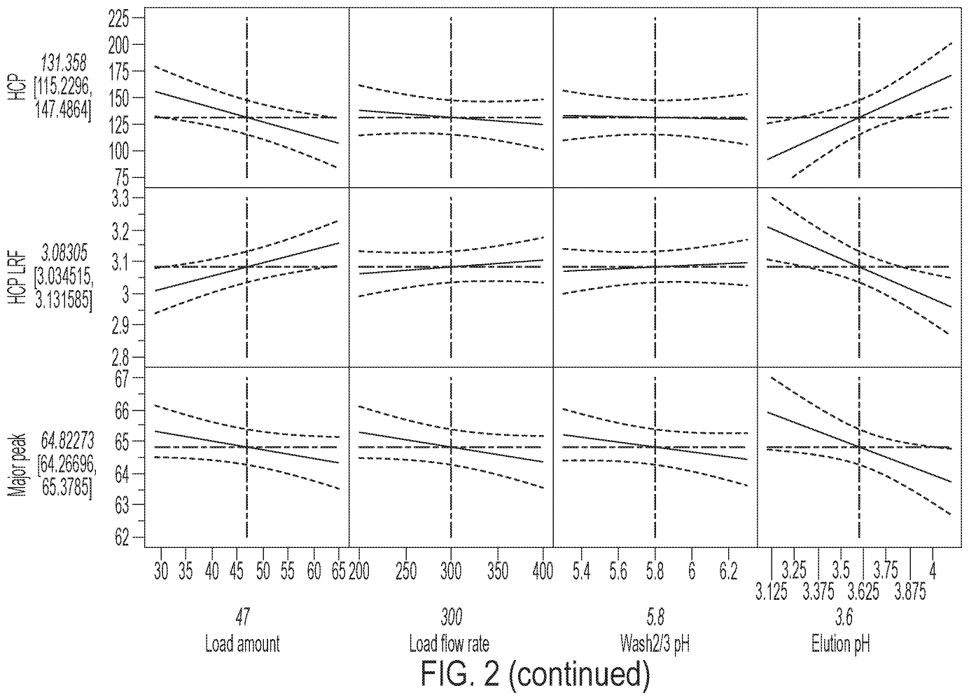

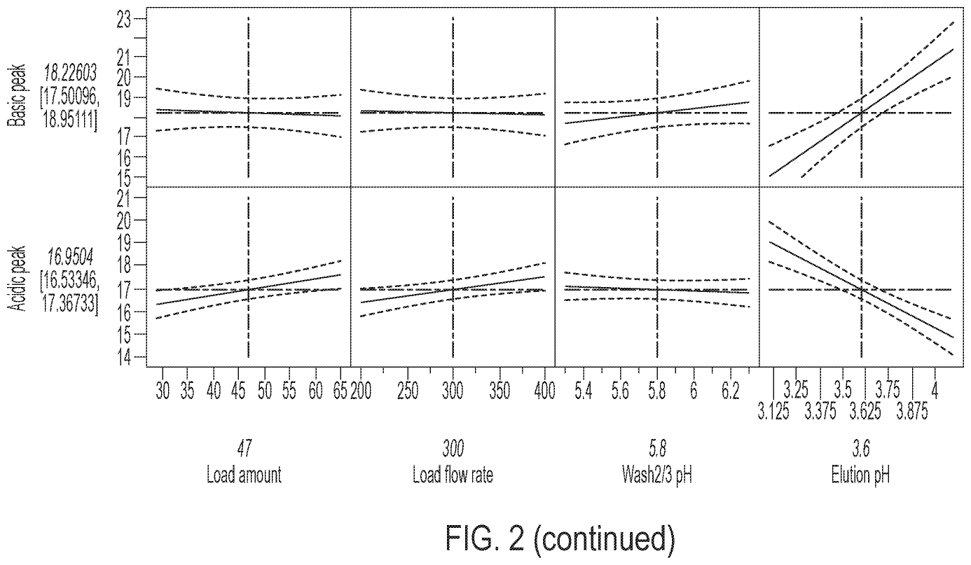

An additional study was performed to identify process parameters associated with Protein A purification that have a significant impact of product quality of vedolizumab. Clarified harvest from GS-CHO cells that recombinantly express vedolizumab was loaded on a MabSelect SuReLX resin (GE Healthcare, Pittsburgh, Pa.). The bound antibody was washed with PBS and sodium citrate buffers prior to elution. Elution pH was evaluated over a range of pH 3.3 to pH 3.9. A sodium citrate buffer was used for elution. The effect of elution buffer pH on level of aggregates (% HMW species) and level of HCP in a purified composition of vedolizumab is described in Table 1 and FIG. 2.

As described in Table 1, a linear regression model showed that elution pH had significant impact on all assay outputs (p<0.05). Although data variation was slightly large on % LMW and HCP, load amount impacted HCP clearance. A combination of load amount and load flow rate had a slight influence on % LMW.

### % Monomer:

Elution pH had significant impact on % Monomer (p<0.05). No other input parameters showed correlation with % Monomer. As the elution pH was raised, the % Monomer was increased.

### % HMW:

Elution pH had significant impact on % HMW (p<0.05). No other input parameters showed correlation with % HMW. As the elution pH was raised, the % HMW decreased.

### % LMW:

Elution pH, and combination of load amount and load flow rate had an impact on % LMW (p<0.05), however, the impact of input parameters on % LMW is considered as minimal.

### HCP:

Elution pH and load amount had a significant impact on HCP with both in ppm and log reduction factor (p<0.05).

TABLE 1
Purification of Vedolizumab using Protein A Affinity
Chromatography—Evaluation of Input Parameters

| | Monomer | HMW | LMW | HCP | HCP |
|---|---|---|---|---|---|
| Elution pH | (%) | (%) | (%) | (ppm) | LRF |
| Load Material | — | — | — | 152590 | — |
| 3.34 | 97.55 | 1.61 | 0.84 | 113 | 3.13 |
| 3.34 | 97.38 | 1.76 | 0.86 | 88 | 3.24 |
| 3.34 | 97.35 | 1.81 | 0.84 | 101 | 3.18 |
| 3.34 | 97.26 | 1.93 | 0.81 | 179 | 2.93 |
| 3.34 | 97.36 | 1.83 | 0.80 | 85 | 3.25 |
| 3.34 | 97.38 | 1.81 | 0.82 | 94 | 3.21 |
| 3.34 | 97.27 | 1.88 | 0.85 | 139 | 3.04 |
| 3.34 | 97.22 | 1.95 | 0.83 | 108 | 3.15 |
| 3.60 | 97.65 | 1.57 | 0.78 | 105 | 3.16 |
| 3.60 | 97.31 | 1.88 | 0.81 | 122 | 3.10 |
| 3.95 | 98.67 | 0.62 | 0.72 | 168 | 2.96 |
| 3.95 | 98.78 | 0.42 | 0.79 | 115 | 3.12 |
| 3.95 | 98.74 | 0.47 | 0.79 | 157 | 2.99 |
| 3.95 | 98.92 | 0.38 | 0.71 | 217 | 2.85 |
| 3.95 | 98.60 | 0.60 | 0.80 | 185 | 2.92 |
| 3.95 | 98.60 | 0.62 | 0.78 | 116 | 3.12 |
| 3.95 | 98.45 | 0.71 | 0.84 | 123 | 3.09 |
| 3.95 | 98.55 | 0.66 | 0.79 | 207 | 2.87 |

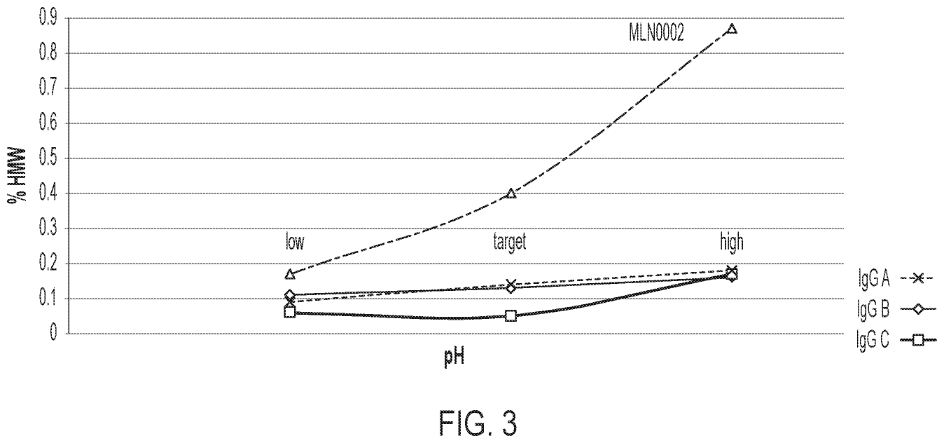

Vedolizumab has characteristics, e.g., high hydrophobicity, that make it unique from other IgG antibodies. FIG. 3 provides a comparison of vedolizumab vs. three other IgG antibodies and provides the amount of aggregate (% HMW) found in the eluate when each antibody (vedolizumab (MLN0002), IgG A, IgG B, or IgG C) was eluted from a cation exchange column (Nuvia S; Bio Rad) using elution buffers having an increasing pH (left to right increase in pH). The performance variability of aggregate clearance for vedolizumab was more variable than the other three tested IgGs at optimal conditions for each. Thus, as observed in FIG. 3, pH can impact aggregation levels during purification of vedolizumab.

### Example 2: Purification of Vedolizumab Using a Hydrophobic HIC Resin

Given the hydrophobic nature of vedolizumab, decreasing HMW aggregates can be challenging during downstream purification. Further, when vedolizumab is produced in mammalian, e.g., CHO, cells, it is also essential to minimize levels of host cell proteins (HCPs). HIC, mixed mode, and anion exchange resins and membranes were screened with high throughput methods for performance. Subsequently, eight HIC resins were tested under various equilibration, load and elution conditions, for the ability of each to both decrease aggregation and minimize HCPs in the purification of vedolizumab. The Toyopearl Hexyl-650C (Tosoh Biosciences) hydrophobic HIC resin was the only resin that was able to demonstrate acceptable aggregate clearance, as well as and minimize HCPs. More specifically, the Hexyl-650C reduced aggregation levels from about 1.5% HMW aggregates to 0.35% under suitable binding conditions (e.g., 0.5 M (NH₄)₂SO₄ at pH 6.7). Other resins tested included butyl-650M, butyl-600M, super butyl-55C, Phenyl-650M, the phenyl-600M, the PPG-600M, and the ether-650M, and were not able to achieve such low levels of aggregate.

Hexyl-650C was the most hydrophobic resin in comparison to other resins that were tested, including ether, PPG, phenyl, and butyl. Hexyl-650C has a mean pore size of about 1,000 A and a mean particle size of about 100 μm.

Further experiments were performed with Hexyl-650C in both bind/elute and flow through mode. For bind/elute experiments, the Hexyl-650C was able to reduce aggregates to about 0.30% HMW, but had a low binding capacity (about 20 mg/ml of resin). In contrast, the flow through mode using Hexyl-650C and vedolizumab provided both a reduction in aggregates and an increased load capacity. Flow through experiments were performed with an initial unadjusted load of 108 mg/ml with 0.2 M sodium chloride in 10 mM sodium phosphate, pH 6.7. These conditions in flow through resulted in a reduction from 1.39% HMW to 0.71% HMW. Increasing the salt, including replacing sodium chloride with potassium phosphate, resulted in an even further improved reduction in aggregates. A reduced load of 67.5 mg/ml of resin using 250 mM potassium phosphate, 50 mM potassium chloride (for equilibration and load adjustment) resulted in an aggregate decrease from about 0.72% HMW to about 0.3% (with 95.5% recovery).

Column-based Design of Experiments (DOE) studies were performed to further evaluate the ability of Hexyl-650C in flow through mode to decrease aggregate levels for purification of vedolizumab. As described in Table 2, low HMW % and low levels of HCP (ppm) were obtained using low pH and increased phosphate load/equilibrium conditions. The experiments in Table 2 were performed using a 60 mg/ml resin load. Low pH conditions all had HMW reduction to values less than or equal to 0.34% from 1.0% HMW. The average recovery of the antibody was about 91.5%. High pH coupled with high potassium phosphate appeared to increase the affinity of the main species of vedolizumab to the resin, reducing recovery. In contrast, higher levels of phosphate coupled with low pH resulted in increased HMW clearance. The low HMW, low HCP, and low residual Protein A leach was observed with low phosphate and low pH (see, e.g., line 12 below). Thus, a high hydrophobicity HIC resin was able to successfully clear aggregate (HMW) vedolizumab to a level below 0.5%.

TABLE 2
DOE Design and Data

| | Potassium | | | | | Residual |
|---|---|---|---|---|---|---|
| Run | Phosphate | | Recovery | HMW | HCP | Protein A |
| # | (M) | pH | (%) | (%) | (ppm) | (ppm) |
| 1 | 0.30 | 5.9 | 99.11 | 0.33 | 0.178 | 0.135 |
| 2 | 0.30 | 7.2 | 68.13 | 0.44 | 0.199 | 0.157 |
| 3 | 0.30 | 6.7 | 103.52 | 0.45 | 0.299 | 0.185 |
| 4 | 0.30 | 6.7 | 103.64 | 0.41 | 0.297 | 0.171 |
| 5 | 0.25 | 6.7 | 93.41 | 0.44 | 0.299 | 0.171 |
| 6 | 0.20 | 5.9 | 93.08 | 0.33 | 0.289 | 0.105 |
| 7 | 0.25 | 6.7 | 96.34 | 0.43 | 0.300 | 0.178 |
| 8 | 0.20 | 6.7 | 93.97 | 0.52 | 0.267 | 0.195 |
| 9 | 0.20 | 6.7 | 83.05 | 0.53 | 0.269 | 0.191 |
| 10 | 0.20 | 7.2 | 102.80 | 0.51 | 0.291 | 0.182 |
| 11 | 0.30 | 5.9 | 91.25 | 0.31 | 0.094 | 0.135 |
| 12 | 0.20 | 5.9 | 91.05 | 0.34 | 0.081 | 0.085 |
| 13 | 0.20 | 7.2 | 96.85 | 0.56 | 0.263 | 0.218 |
| 14 | 0.30 | 7.2 | 73.86 | 0.38 | 0.093 | 0.149 |

### Example 3: Effect of Column Load, Elution Buffer pH and Conductivity on Purification of Vedolizumab Using a Mixed Mode Chromatography Resin

A production CHO cell line expressing high vedolizumab antibody titers (≥5.0 g/L) was generated, requiring the development of a purification process designed to accommodate large amounts of this highly hydrophobic antibody.

Capto Adhere ImpRes is a mixed mode (MXM) chromatography resin which has a strong anion exchange, hydrogen bonding, and hydrophobic interaction functionality on a smaller bead size, allowing for improved impurity removal and increased capacity.

The Capto Adhere ImpRes mixed mode resin operated in flow-through mode was able to purify vedolizumab, but the yield and level of impurity removal were lower than desired. Pre-characterization experiments conducted using Capto Adhere ImpRes in bind-elute mode indicated significant loss of step yield and/or impurity removal capacity related to three process input parameters: resin load capacity, elution buffer pH, and elution buffer conductivity. The present Example describes a study that was designed to further examine the effects of variation in these parameters on the performance of Capto Adhere ImpRes for purification of vedolizumab, and their impact on various product quality attributes.

### Materials and Methods

Clarified cell culture harvest was loaded on a Capto Adhere ImpRes (GE Healthcare, Chicago, Ill., USA) chromatography column after purification over Protein A. The mixed mode resin was washed using a sodium phosphate buffer at pH 7.8, and the antibody was eluted from the column under varying conditions, as described below.

Samples were submitted for analysis immediately by SEC, stored at 2-8° C., and processed within 1 week. The remaining assays (CEX, CHO HCP ELISA) were performed using frozen retains (−80° C.). The methods used for analysis are listed below in TABLE Table 3, and are described in detail below. The load materials sampled after queued runs were analyzed to confirm that there is no substantial change in the quality attributes of the load material.

TABLE 3
Analytical Methods

| Description | Quality of Interest |
|---|---|
| Concentration by UV Absorbance | Protein concentration |
| at 280 nm (Solo VPE) | |
| Size Exclusion Chromatography | Aggregates, fragments |
| Cation Exchange Chromatography | Charge variants |
| CHO HCP ELISA | Residual CHO HCP content |
| pH Measurement | Load or Eluate pH |
| Conductivity Measurement | Load or Eluate Conductivity |

Experimental Design

A full factorial design was utilized on the resin load amount, elution buffer pH and elution buffer conductivity at three levels. A sodium citrate elution buffer was used in these experiments. The resulting experimental design included thirty runs with three center point conditions. An additional center point condition is part of the experimental design designated with DOE pattern 222 in Table 5. All other process input parameters were kept at center point conditions. Elution buffer sodium chloride concentration was used to design the experiment and elution buffer conductivity measurements were used as input parameter values for statistical analysis. The parameter ranges studied and the outline of the design are described in Table 4 and TABLE 5 respectively.

TABLE 4
Evaluated Parameter Ranges

| | | Evaluated | |
|---|---|---|---|
| Parameter | Units | Ranges | Notes |
| Protein Load to Volume | g/L | 53-77 | — |
| of Resin | | | |
| Elution Buffer pH | pH | 3.8-4.4 | — |
| Elution Buffer | mS/cm | 19.7-28.9 | NaCl conc. range |
| Conductivity | | | of 160-240 mM |

TABLE 5
Experimental Design

| | | | | Protein |
|---|---|---|---|---|
| | | | Elution | Load to |
| | | Elution | Buffer | Resin |
| | DOE | Buffer | [NaCl] | Volume, |
| Run # | Pattern | pH | (mM) | (g/L) |
| 1 | 311 | 4.4 | 160 | 53 |
| 2 | 112 | 3.8 | 160 | 65 |
| 3 | 223 | 4.1 | 200 | 77 |
| 4 | 313 | 4.4 | 160 | 77 |
| 5 | 122 | 3.8 | 200 | 65 |
| 6 | 222 | 4.1 | 200 | 65 |
| 7 | 113 | 3.8 | 160 | 77 |
| 8 | 232 | 4.1 | 240 | 65 |
| 9 | 131 | 3.8 | 240 | 53 |
| 10 | 212 | 4.1 | 160 | 65 |
| 11 | 332 | 4.4 | 240 | 65 |
| 12 | 323 | 4.4 | 200 | 77 |
| 13 | 331 | 4.4 | 240 | 53 |
| 14 | 312 | 4.4 | 160 | 65 |
| 15 | 000 | 4.1 | 200 | 65 |
| 16 | 333 | 4.4 | 240 | 77 |
| 17 | 121 | 3.8 | 200 | 53 |
| 18 | 000 | 4.1 | 200 | 65 |
| 19 | 322 | 4.4 | 200 | 65 |
| 20 | 231 | 4.1 | 240 | 53 |
| 21 | 000 | 4.1 | 200 | 65 |
| 22 | 111 | 3.8 | 160 | 53 |
| 23 | 221 | 4.1 | 200 | 53 |
| 24 | 123 | 3.8 | 200 | 77 |
| 25 | 211 | 4.1 | 160 | 53 |
| 26 | 133 | 3.8 | 240 | 77 |
| 27 | 321 | 4.4 | 200 | 53 |
| 28 | 213 | 4.1 | 160 | 77 |
| 29 | 132 | 3.8 | 240 | 65 |
| 30 | 233 | 4.1 | 240 | 77 |

DOE pattern (3): upper level, (2): medium level, (1): lower level; (0): center point for each input parameter range. Experiment was designed using NaCl concentration as input parameter and actual conductivity measurements were used in statistical analysis.

Calculations

% HMW clearance=(1−(eluate HMW/load HMW))*100

Logarithmic reduction factor (LRF) and was determined as:

LRF=log₁₀ {Residual_{load}/Residualₑₗᵤₐₜₑ}

[Residualₑₗᵤₐₜₑ] is the concentration of CHO HCP or Protein A in the eluate and [Residual_{load}] is the concentration of CHO HCP or Protein A in the load of the same run, both concentrations in units of ppm, relative to the corresponding MLN0002 concentrations.

Statistical Analysis

The product quality indicating assay outputs and KPIs for all experiments in the design were analyzed using JMP 11 statistical software (SAS Institute, Cary, N.C.). Each response was analyzed via fitting to a linear model, shown in Equation 1:

$$Generic Linear Model Regression \\ {y}_{ι ι} = {β}_{0} + {\sum }_{i = 1}^{k} {β}_{{i}^{{X}_{i ι ι}}} \qquad Equation 1$$

where yᵤ is the response at the u^{th} observation, xᵢᵤ are the independent variables, and the various β terms are the model coefficient estimates.

In statistical analysis and modeling, fitting a relatively small data set to a model containing a relatively large number of potential inputs often gives rise to over-fitting. A hallmark of over-fitting is a model with a high R² value but several scientifically meaningless (and statistically insignificant) terms. To determine the best statistically significant model, while avoiding over-fitting, each model was developed using forward regression of the input parameters and the response of interest. The stopping rule for the regression was a p-value threshold, in which an input parameter was incorporated into the model if its p-value was ≤0.05. This algorithm for analysis generated models that (i) achieve the highest possible R² values, (ii) include as few input parameters as possible, and (iii) describe behavior that is physically possible for antibodies undergoing multi-modal chromatography processing (even if such findings appear to disagree with initial technical expectations).

### Results and Discussion

Experimental results are described in Tables 6 and 7, which contain the parameter estimates, corresponding p-values, and R² values for the model of each response determined through statistical analysis.

TABLE 6
Summary of Statistical Model, Prediction Expression Coefficients, and p-values—Process
Performance

| | | | | Eluate | Eluate |
|---|---|---|---|---|---|
| | % | | | Cond. | Conc. |
| Parameter | Recovery | Eluate CV | Eluate pH | (mS/cm) | (g/L) |
| Intercept | 210.3775 | −20.0321 | 4.3495 | −21.1738 | 117.7005 |
| | (p < 0.0001) | (p < 0.0001) | (p < 0.0001) | (p < 0.0001) | (p < 0.0001) |
| Elution pH | −29.6435 | 5.3704 | 0.1184 | 4.6490 | −25.4174 |
| | (p < 0.0001) | (p < 0.0001) | (p = 0.0278) | (p < 0.0001) | (p < 0.0001) |
| Elution Conductivity | −0.3489 | 0.0992 | −0.0169 | 1.0025 | −0.5757 |
| (mS/cm) | (p = 0.0189) | (p < 0.0001) | (p = 0.0003) | (p < 0.0001) | (p < 0.0001) |
| Load Amount (g/L of | 0.1528 | — | 0.0027 | — | 0.2422 |
| Resin) | (p = 0.0021) | | (p = 0.0462) | | (p < 0.0001) |
| (Elution pH- 4.0998) * | 0.4539 | — | — | — | −0.3724 |
| (Load Amount, g/L Resin - | (p = 0.0170) | | | | (p = 0.0112) |
| 65) | | | | | |
| (Elution pH- | — | −0.1184 | 0.0711 | — | 1.8872 |
| 4.0998)*(Elution Cond., | | (p = 0.0098) | (p = 0.0001) | | (p = 0.0001) |
| mS/cm-24.216) | | | | | |
| R² | 0.930 | 0.986 | 0.642 | 0.973 | 0.955 |

TABLE 7
Summary of Statistical Model, Prediction Expression Coefficients, and p-values-Product Quality (SEC and CEX)

| | SEC | | | | | | |
|---|---|---|---|---|---|---|---|
| | % HMW | | | | CEX | | |
| Parameter | Clearance | % HMW | % LMW | % Monomer | % Acidic | % Basic | % Main |
| Intercept | −258.6155 | 5.0702 | 0.3877 | 94.5375 | 18.1046 | 6.8334 | 73.4677 |
| | (p < 0.0001) | (p < 0.0001) | (p = 0.0055) | (p < 0.0001) | (p < 0.0001) | (p < 0.0001) | (p < 0.0001) |
| Elution pH | 72.4266 | −1.0151 | 0.1533 | 0.8675 | −1.1010 | 1.3884 | — |
| | (p < 0.0001) | (p < 0.0001) | (p < 0.0001) | (p < 0.0001) | (p = 0.0020) | (p < 0.0001) | |
| Elution | 0.9806 | −0.0163 | — | 0.0145 | — | 0.0758 | −0.0586 |
| Conductivity | (p = 0.0003) | (p < 0.0001) | | (p = 0.0005) | | (p = 0.0012) | (p = 0.0465) |
| (mS/cm) | | | | | | | |
| Load Amount | −0.2668 | 0.0039 | −0.0036 | — | — | — | — |
| (g/L of Resin) | (p = 0.0016) | (p = 0.0015) | (p < 0.0001) | | | | |
| (Elution pH- | — | — | −0.0065 | — | — | −0.0671 | — |
| 4.0998) * (Load | | | (p = 0.0354) | | | (p = 0.0182) | |
| Amount, g/L | | | | | | | |
| Resin-65) | | | | | | | |
| (Elution pH- | — | 0.0286 | — | −0.0323 | — | — | — |
| 4.0998)*(Elution | | (p = 0.0462) | | (p = 0.0341) | | | |
| Cond., mS/cm- | | | | | | | |
| 24.216) | | | | | | | |
| R² | 0.962 | 0.962 | 0.684 | 0.940 | 0.293 | 0.680 | 0.134 |

Effects of Elution Buffer pH and Conductivity on Yield of Antibody

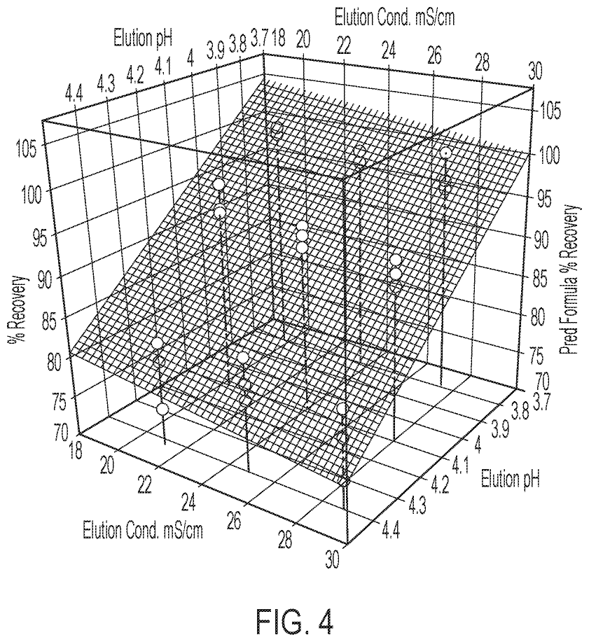

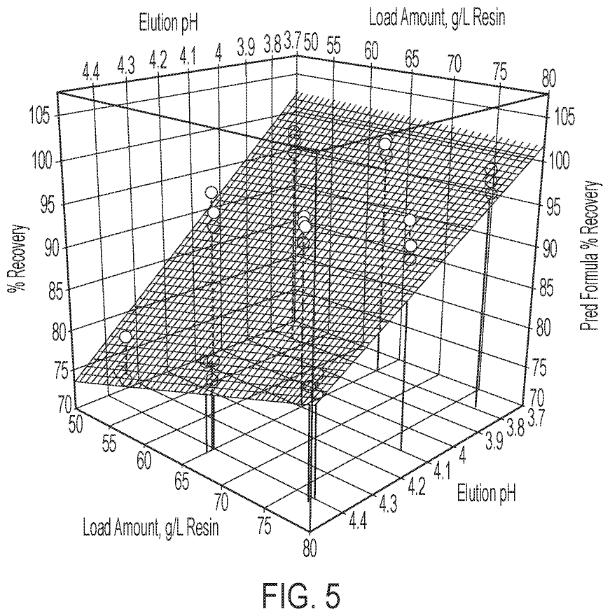

The effects of elution buffer pH and elution buffer conductivity on step recovery or yield of vedolizumab are described in FIGS. 4 and 5. As described in FIGS. 4 and 5, the observed step recovery data fit to a linear regression model well, as indicated with a R² value of 0.930.

FIGS. 4 and 5 show plots of Capto Adhere ImpRes step recovery versus elution buffer pH and conductivity or load amount. The recovery was significantly influenced by elution buffer pH (p<0.0001) and resin load amount (p=0.0021) and to a lesser extent by elution buffer conductivity (p=0.0189). Elution buffer pH of ˜4.40 at any level of elution buffer conductivity and load amount resulted in step yields of ≤83.91% (Run 1, 4, 11-14, 16, 19, and 27). Low resin load amounts had a negative impact on recovery. Breakthrough was observed in all runs with load amounts of 77 g/L of resin during the wash step following sample loading. The influence of load amount was dependent on elution buffer pH (p=0.0170). At low elution buffer pH (i.e., pH 3.8), load amount had no practical impact on recovery, whereas, as the elution buffer pH increased, reduced resin load resulted in lower recoveries (73.36-78.84% at 53 g/L resin vs. 81.94-83.91% at 77 g/L resin load amounts at elution buffer pH ˜4.4). The elution buffer conductivity only had a minor impact on recovery according to experimental results and model predictions. The lowest recovery of 73.36% was observed at elution buffer pH of 4.40, elution buffer conductivity of 28.89 mS/cm, and load amount of 53 g/L resin (Run 13, predicted as 76.22% by the model).

Thus, as described in FIGS. 4 and 5, yield of vedolizumab is increased when mixed mode chromatography resin is used with an elution buffer having optimized pH and conductivity.

Effects of Elution Buffer pH and Conductivity on HMW Aggregates

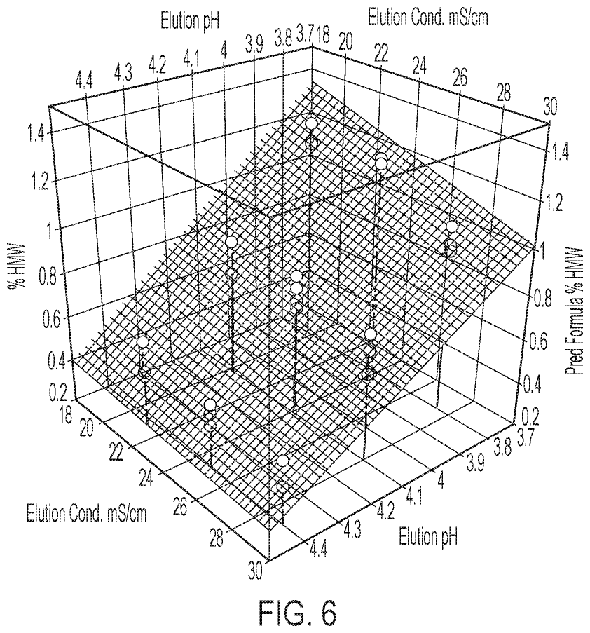

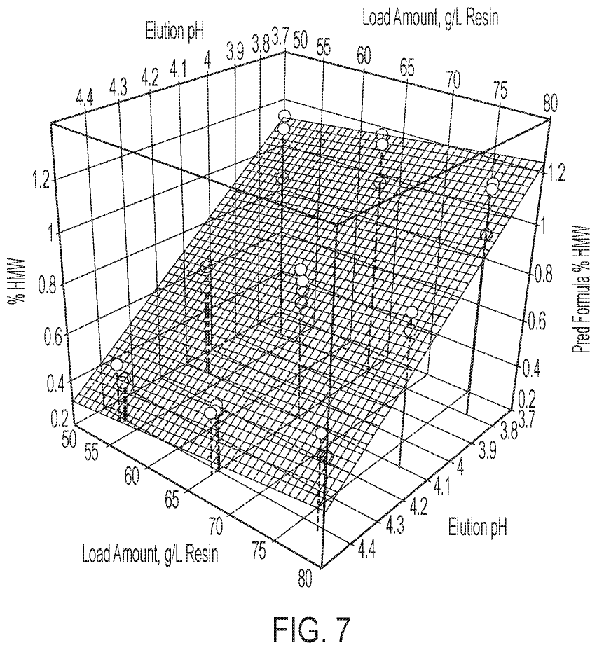

The effects of elution buffer pH and conductivity on level of aggregates are described in FIGS. 6 and 7, which show the impact of input parameters on HMW amounts.

The amount of HMW species in the eluate is impacted by the elution buffer pH (p<0.0001), elution buffer conductivity (p<0.0001), and load amount (p=0.0015) according to the linear regression model (R²=0.962). Increased elution pH and conductivity reduces the amount of HMW species in the eluate, while increased load amount leads to higher levels. The model also predicts statistically significant (p=0.0462) interactions between elution buffer pH and conductivity.

As described in FIGS. 6 and 7 below, HMW species content of ˜1% or higher was observed with elution buffer pH of ˜3.80. The elution pH of 3.80, elution conductivity of 19.67 mS/cm (160 mM NaCl) and load amount of 65 g/L of resin (Run 2) resulted in the highest HMW content of 1.23% (predicted by the model as 1.19%). The predicted worst case HMW content was 1.24% at 77 g/L under the same elution buffer conditions.

According to the linear regression model, elution buffer pH (p<0.0001), elution buffer conductivity (p=0.0003) and load amount (p=0.0016) each have a statistically significant impact on HMW clearance capacity. Some level of clearance (12.50-72.79%) was achieved at any condition evaluated in this study. Among these inputs, elution pH had the highest impact. Increased elution buffer pH improved the HMW clearance, which is the opposite of the effect it had on recovery. Increased elution buffer conductivity and reduced load amount increased the HMW clearance, in accordance with the model.

Thus, as described in FIGS. 6 and 7, the level of aggregates (% HMW species) in a purified composition of vedolizumab can be modulated by selection of the elution buffer pH and elution buffer conductivity used to elute the antibody from a mixed mode chromatography resin. In addition, the level of aggregates can be reduced when a mixed mode chromatography resin is used with an elution buffer having elevated pH and/or elevated conductivity.

### Example 4: Effect of Elution Buffer on Purification of Vedolizumab Using a Cation Exchange (CEX) Resin

Cation exchange (CEX) chromatography was also explored as a means to further reduce the level of aggregates in a vedolizumab preparation. Described herein is a study focused on adapting CEX chromatography using the Nuvia HR-S resin (Bio-Rad, Hercules, Calif., USA) to the purification of vedolizumab, with a specific focus on the reduction of aggregate levels from this hydrophobic antibody. Elution conditions for a CEX process operated in bind/elute mode were assessed. A design of experiment (DoE) approach was employed to evaluate the impact of several parameters, including elution buffer pH and elution buffer conductivity, on process outputs.

### Materials and Methods

The load materials and analytical methods used in the present study are similar to that described in Example 3 above.

Experimental Design

An initial screening study and preliminary risk assessment identified elution buffer pH and elution buffer conductivity as having a known or potential impact on Nuvia HR-S process performance outputs (PPOs), when the resin is operated in bind/elute mode. The present study was conducted to characterize the effect of these process parameters. The studies parameter ranges and the experimental design are listed in Table 8 and Table 9, respectively. The elution buffer conductivity was varied by modulating the concentration of sodium chloride (NaCl), and the evaluated NaCl range is provided in Table 8.

TABLE 8
Study Process Parameters

| Parameter | Evaluated Range | |
|---|---|---|
| Load Amount | 30-65 | g mAb/L resin |
| Elution Buffer pH | 5.1-5.7 | |
| Elution Buffer | 10.59-16.08 | mS/cm |
| Conductivity | | |
| Elution Buffer [NaCl] | 70-110 | mM |

TABLE 9
Experimental Design

| | Elution | Elution Buffer | Load Amount |
|---|---|---|---|
| Run # | Buffer pH | [NaCl] (mM) | (g mAb/L resin) |
| 1 | 5.1 | 90 | 65 |
| 2 | 5.4 | 90 | 47.5 |
| 3 | 5.4 | 90 | 47.5 |
| 4 | 5.1 | 70 | 47.5 |
| 5 | 5.1 | 110 | 47.5 |
| 6 | 5.4 | 110 | 65 |
| 7 | 5.4 | 90 | 47.5 |
| 8 | 5.4 | 90 | 65 |
| 9 | 5.7 | 110 | 30 |
| 10 | 5.1 | 70 | 30 |
| 11 | 5.4 | 70 | 47.5 |
| 12 | 5.4 | 90 | 30 |
| 13 | 5.4 | 70 | 30 |
| 14 | 5.7 | 70 | 65 |
| 15 | 5.7 | 90 | 65 |
| 16 | 5.1 | 90 | 30 |
| 17 | 5.7 | 70 | 47.5 |
| 18 | 5.4 | 70 | 65 |
| 19 | 5.1 | 70 | 65 |
| 20 | 5.7 | 110 | 65 |
| 21 | 5.7 | 90 | 47.5 |
| 22 | 5.7 | 90 | 30 |
| 23 | 5.7 | 70 | 30 |
| 24 | 5.7 | 110 | 47.5 |
| 25 | 5.1 | 90 | 47.5 |
| 26 | 5.4 | 110 | 30 |
| 27 | 5.1 | 110 | 65 |
| 28 | 5.4 | 110 | 47.5 |
| 29 | 5.1 | 110 | 30 |
| 30 | 5.2 | 90 | 30 |
| 31 | 5.2 | 70 | 47.5 |
| 32 | 5.2 | 70 | 30 |
| 33 | 5.2 | 90 | 65 |
| 34 | 5.2 | 70 | 65 |
| 35 | 5.2 | 90 | 47.5 |
| 36 | 5.6 | 80 | 38.75 |
| 37 | 5.6 | 80 | 56.25 |
| 38 | 5.6 | 100 | 38.75 |
| 39 | 5.6 | 100 | 56.25 |
| 40 | 5.54 | 92 | 65 |
| 41 | 5.5 | 92 | 65 |
| 42 | 5.6 | 92 | 65 |

### Results and Discussion

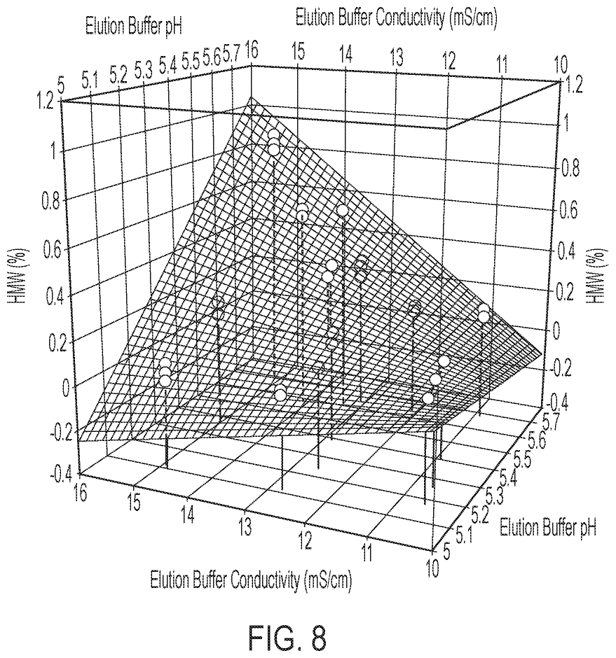

The effects of elution buffer pH and elution buffer conductivity on Nuvia HR-S process performance outputs (PPOs) are described in FIGS. 8-13.

Effects of Elution Buffer pH and Conductivity on HMW Aggregates

The effects of elution buffer pH and conductivity on level of aggregates are described in FIGS. 8-10, which show the impact of input parameters on HMW amounts.

As described in FIGS. 8-10, variations in the eluate HMW, monomer, and LMW were 0.01 to 0.88%, 98.33 to 99.27%, and 0.62 to 1.35%, respectively. The model for HMW contains strong linear dependences on elution buffer pH and conductivity, in addition to an interaction term containing both parameters. The model surface (as shown in FIG. 8) indicates that HMW is lowest at the extreme low values of elution buffer pH and conductivity and highest at the extreme high values of elution buffer pH and conductivity.

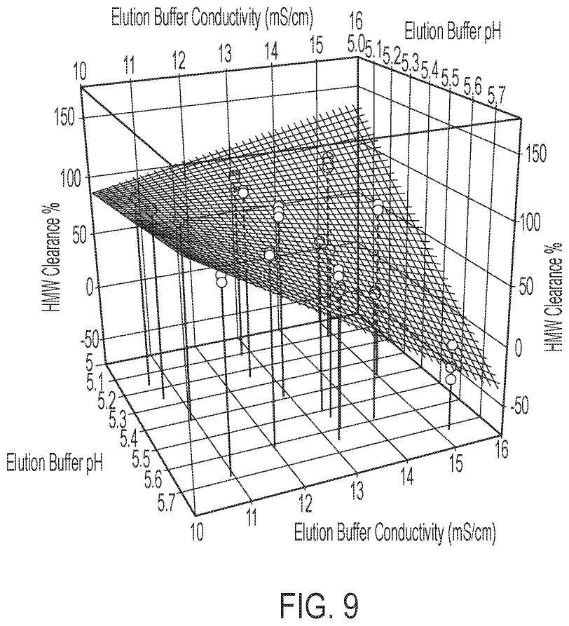

HMW clearance was determined for each run to account for variation in load material HMW content throughout the study. As with eluate HMW, HMW clearance varied dramatically across the study (−30.65 to 98.61%), and the HMW clearance model contains linear and interaction terms for elution buffer pH and conductivity. FIG. 9 displays the model behavior: while the highest HMW clearance values were achieved at decreased elution buffer pH and conductivity conditions, many tested conditions demonstrated >70% HMW clearance. However, the negative HMW clearance values reported for runs 9, 20, and 24 (−1.18%, −20.55%, and −30.65%, respectively) indicate that substantial variation in aggregate removal was observed across the broad range of conditions evaluated, and that some conditions may generate, rather than remove, aggregate species.

Runs 40 to 42 employed Capto Adhere ImpRes eluate as load material, which contained aggregate levels higher than those typically used in the load material during processing on CEX at center point conditions. The models for HMW and HMW clearance showed that increasing elution buffer pH and conductivity produce eluate with increased HMW content. The conditions selected for runs 40 to 42 aimed to probe potential elution conditions using “worst case” aggregate levels for the CEX load material. For elution buffer pH values of 5.50 and 5.54 and elution buffer conductivity of 13.40 mS/cm, the resulting eluate HMW was 0.31 to 0.34%. However, the eluate HMW increased to 0.59% when employing a pH 5.60 elution buffer (while conductivity remained unchanged). At that aggregate level, further processing would risk the failure of the vedolizumab acceptance criterion for HMW.

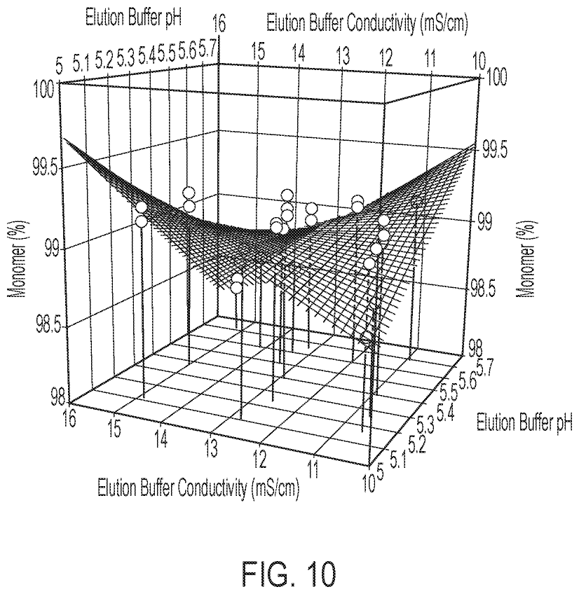

LMW was found to decrease linearly with respect to increases in elution buffer pH or elution buffer conductivity. The model also contained interaction terms for elution buffer pH/elution buffer conductivity, elution buffer pH/load amount, and elution buffer conductivity/load amount. As in the model for HMW, the monomer model contained strong dependences on elution buffer pH and conductivity in the form of linear and interaction terms. The model surface (shown as a saddle function in FIG. 10) indicates that the lowest monomer is achieved at the combined extreme high conditions of elution buffer pH and conductivity.

Thus, as described in FIGS. 8-10, the elution buffer pH and conductivity can be used to modulate the level of aggregates (% HMW species) in a composition comprising vedolizumab that is purified using a CEX resin. In addition, as shown in FIGS. 8-10, the level of aggregates in a purified composition of vedolizumab can be reduced when a CEX resin is used with an elution buffer having reduced pH and/or reduced conductivity.

Effects of Elution Buffer pH and Conductivity on Basic Isoform Species

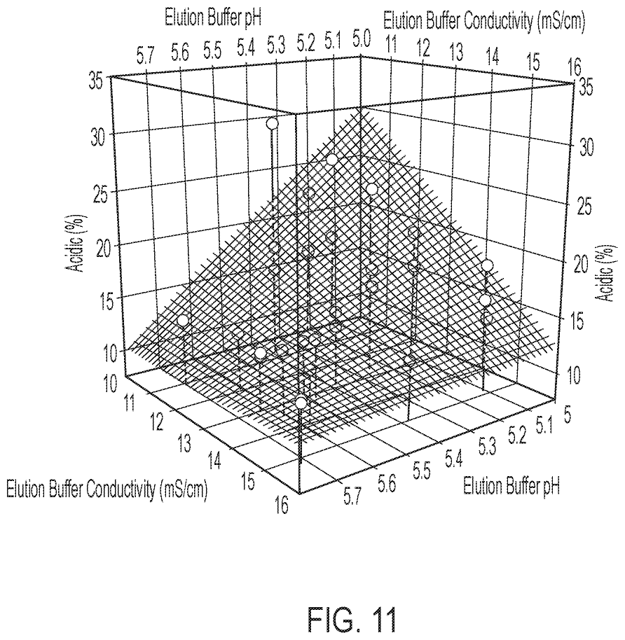

The effects of elution buffer pH and conductivity on level of basic isoform species are described in FIGS. 11-13, which show the impact of input parameters on the content of acidic, major and basic isoform species.

As described in FIGS. 11-13, the acidic, major, and basic content results ranged from 12.49 to 30.27%, 64.32 to 73.82%, and 5.42 to 18.04%, respectively. The model for acidic content contains linear terms for elution buffer pH, elution buffer conductivity, and load amount, in addition to interaction terms for all three parameters. Elution buffer pH and elution buffer conductivity most strongly influence acidic content. As seen in the model surface in FIG. 11, the highest acidic content was achieved when operating at the combined extreme low values for elution buffer pH and conductivity.

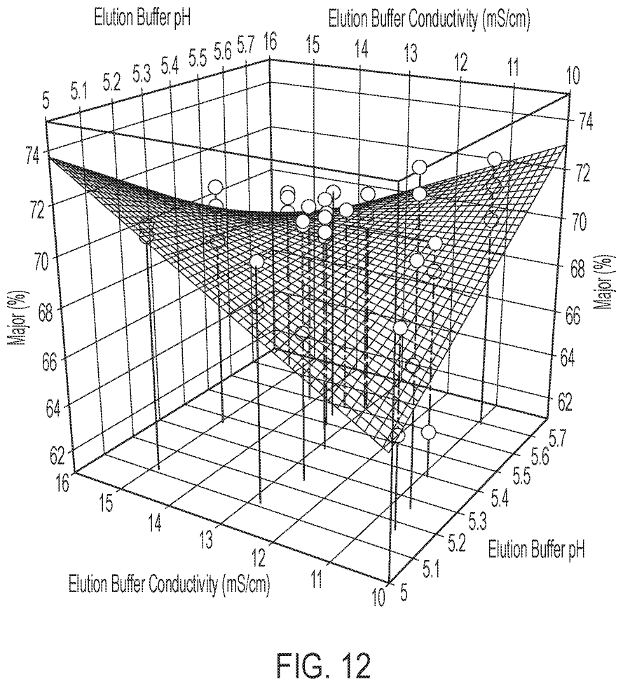

The model developed for major isoform content contains interaction terms between elution buffer pH, elution buffer conductivity, and load amount, where the elution buffer pH/elution buffer conductivity interaction exhibits the greatest influence on model behavior. As shown in FIG. 12, the highest major isoform content was achieved at the combined highest elution buffer conductivity and lowest elution buffer pH; the lowest major isoform content is predicted for the combined extreme low values of elution buffer pH and conductivity.

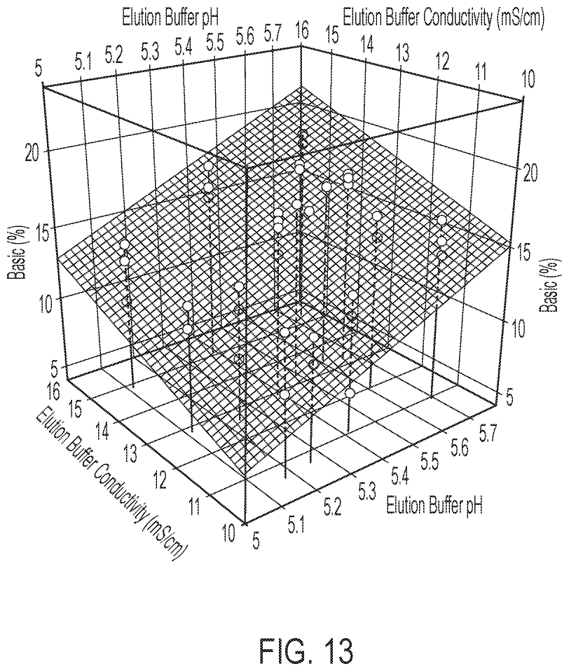

Linear terms for elution buffer pH, elution buffer conductivity, and load amount most strongly influence eluate basic isoform content; interaction terms show minor contributions. As seen in the model surface in FIG. 13, basic isoform content increased in response to increased elution buffer pH and elution buffer conductivity.

Thus, as described in FIGS. 11-13, elution buffer pH and conductivity can be used to modulate the charged isoform distribution in a composition comprising vedolizumab that is purified using a CEX resin. As shown in FIG. 11, the level of acidic isoform species in a purified composition of vedolizumab can be reduced when a CEX resin is used with an elution buffer having increased pH and/or increased conductivity. In addition, as shown in FIG. 13, the level of basic isoform species in a purified composition of vedolizumab can be reduced when a CEX resin is used with an elution buffer having reduced pH and/or reduced conductivity.

### Example 5: Determination of Product Quality Attributes

The following analytical assays and methods were used in the foregoing examples to determine the product quality attributes of vedolizumab.

Cation exchange chromatography (CEX) fractionates vedolizumab antibody species (major isoform, basic species, and acidic species) according to overall surface charge. After dilution to low ionic strength using mobile phase, the test sample is injected onto a Dionex Pro-Pac™ WCX-10 column (Thermo Fisher Scientific, Waltham, Mass. (USA)) equilibrated in 10 mM sodium phosphate, pH 6.6, and eluted using a sodium chloride gradient in the same buffer. Protein elution is monitored at 280 nm and peaks are assigned to acidic, basic, or major isoforms categories. The percent major isoform, the sum of percent acidic species, and the sum of percent basic species are reported. The major isoform retention time of the sample is compared with that of the reference standard to determine the conformance.

Size-exclusion chromatography (SEC) is used to determine the purity of vedolizumab. Reference standard and test samples (75 μg) are analyzed using two G3000 SWxl columns (Tosoh Bioscience, King of Prussia, Pa. (USA)) connected in tandem and an isocratic phosphate-sodium chloride buffer system, pH 6.8. The method provides separation of antibody monomer from high molecular weight (HMW) species as well as low molecular weight (LMW) degradation products. Elution of protein species is monitored at 280 nm. The main peak (monomer) and the total peak area are assessed to determine purity. The purity (%) of the sample (calculated as % monomer) and the % aggregate are reported.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

SEQUENCE TABLE

| SEQ | | |
|---|---|---|
| ID NO: | DESCRIPTION | SEQUENCE |
| 1 | Heavy chain | QVQLVQSGAEVKKPGASVKVSCKGSGYTFTSYWMHWVRQAPGQR |
| | (HC) variable | LEWIGEIDPSESNTNYNQKFKGRVTLTVDISASTAYMELSSLRSEDT |
| | region (amino | AVYYCARGGYDGWDYAIDYWGQGTLVTVSS |
| | acid) | |
| 2 | HC CDR1 | SYWMH |
| | (amino acid) | |
| 3 | HC CDR2 | EIDPSESNTNYNQKFKG |
| | (amino acid) | |
| 4 | HC CDR3 | GGYDGWDYAIDY |
| | (amino acid) | |
| 5 | Light chain (LC) | DVVMTQSPLSLPVTPGEPASISCRSSQSLAKSYGNTYLSWYLQKPGQ |
| | variable region | SPQLLIYGISNRFSGVPDRFSGSGSGTDFTLKISRVEAEDVGVYYCLQ |
| | (amino acid) | GTHQPYTFGQGTKVEIK |
| 6 | LC CDR1 (amino | RSSQSLAKSYGNTYLS |
| | acid) | |
| 7 | LC CDR2 (amino | GISNRFS |
| | acid) | |
| 8 | LC CDR3 (amino | LQGTHQPYT |
| | acid) | |
| 9 | Heavy chain | QVQLVQSGAEVKKPGASVKVSCKGSGYTFTSYWMHWVRQAPGQR |
| | amino acid | LEWIGEIDPSESNTNYNQKFKGRVTLTVDISASTAYMELSSLRSEDT |
| | sequence | AVYYCARGGYDGWDYAIDYWGQGTLVTVSSASTKGPSVFPLAPSS |
| | | KSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHT |
| | | CPPCPAPELAGAPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALPAPIEKTISKAKGQPREPQVYTLPPSRDELTKNQV |
| | | SLTCLVKGFYPSDIAVEWESNGQPENNYKTTPPVLDSDGSFFLYSKL |
| | | TVDKSRWQQGNVFSCSVMHEALHNHYTQKSLSLSPGK |
| 10 | Light chain | DVVMTQSPLSLPVTPGEPASISCRSSQSLAKSYGNTYLSWYLQKPGQ |
| | amino acid | SPQLLIYGISNRFSGVPDRFSGSGSGTDFTLKISRVEAEDVGVYYCLQ |
| | sequence | GTHQPYTFGQGTKVEIKRTVAAPSVFIFPPSDEQLKSGTASVVCLLN |
| | | NFYPREAKVQWKVDNALQSGNSQESVTEQDSKDSTYSLSSTLTLSK |
| | | ADYEKHKVYACEVTHQGLSSPVTKSFNRGEC |

1. A method for obtaining a composition comprising an anti-α4β7 antibody from a liquid solution comprising the anti-α4β7 antibody and one or more impurities, said method comprising
contacting a matrix comprising Protein A with the liquid solution comprising the anti-α4β7 antibody and one or more impurities, such that the anti-α4β7 antibody binds to the Protein A;
washing the matrix comprising Protein A with a wash solution; and
eluting the anti-α4β7 antibody from the matrix comprising Protein A by contacting the matrix with an elution solution having a pH of 3.2 to 4, such that a composition comprising the anti-α4β7 antibody is obtained,
wherein the anti-α4β7 antibody is a humanized antibody, is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

2. The method of claim 1, wherein the composition comprising the anti-α4β7 antibody comprises less than 1% high molecular weight (HMW) aggregate.

3-4. (canceled)

5. The method of claim 1, wherein the wash solution has a pH of about 7.

6. The method of claim 1, wherein:
the elution solution comprises citric acid; or
the elution solution has a pH of 3.2 to 3.7 or 3.3 to 3.8.

7. (canceled)

8. A method for obtaining a composition comprising an anti-α4β7 antibody from a liquid solution comprising an anti-α4β7 antibody and one or more impurities, said method comprising
contacting a solution comprising an anti-α4β7 antibody and at least one impurity with a hydrophobic interaction chromatography (HIC) resin under conditions that allow flow through of the anti-α4β7 antibody through the HIC resin, such that a composition comprising the anti-α4β7 antibody is obtained,
wherein the HIC resin is characterized as a high hydrophobic HIC resin,
wherein the anti-α4β7 antibody is a humanized antibody, is an IgG1 antibody, comprises a heavy chain variable region comprising a CDR3 domain as set forth in SEQ ID NO: 4, a CDR2 domain as set forth in SEQ ID NO: 3, and a CDR1 domain as set forth in SEQ ID NO: 2; and comprises a light chain variable region comprising a CDR3 domain as set forth in SEQ ID NO: 8, a CDR2 domain as set forth in SEQ ID NO: 7, and a CDR1 domain as set forth in SEQ ID NO: 6.

9. The method of claim 8, wherein the composition comprising the anti-α4β7 antibody, comprises less than 0.6% HMW aggregate.

10. The method of claim 8, wherein the HIC resin is equilibrated with a phosphate buffer having a pH of less than about 7.2.

11. (canceled)

12. The method of claim 8, wherein the resin load is about 55 to 75 mg/ml.

13. The method of claim 8, wherein the composition comprises less than about 0.22 ppm residual protein A and/or the composition contains less than about 0.3 ppm host cell protein (HCP).

14-16. (canceled)

17. A method for producing a composition comprising an anti-α4β7 antibody from a liquid solution comprising the anti-α4β7 antibody and one or more impurities, said method comprising
contacting the liquid solution comprising the anti-α4β7 antibody and one or more impurities with a mixed mode chromatography resin, such that the anti-α4β7 antibody binds to the resin;
washing the mixed mode chromatography resin with a wash solution; and
eluting the anti-α4β7 antibody from the mixed mode chromatography resin by contacting the resin with an elution solution having a pH at or above pH 3.9, such that a composition comprising the anti-α4β7 antibody is obtained,
wherein the anti-α4β7 antibody comprises a heavy chain variable region set forth in SEQ ID NO:1, and a light chain variable region set forth in SEQ ID NO:2.

18. The method of claim 17, wherein the composition comprising the anti-α4β7 antibody, comprises less than 1% HMW aggregate.

19-20. (canceled)

21. The method of claim 17, wherein:
the elution solution has a conductivity of 30 mS/cm or less; or
the elution solution has a conductivity of about 20 mS/cm to about 30 mS/cm.

22. (canceled)

23. The method of claim 17, wherein the elution solution comprises NaCl at a concentration of about 160 mM to about 240 mM.

24. (canceled)

25. The method of claim 17, wherein the method further comprises purifying the anti-α4β7 antibody using a cation exchange (CEX) resin.

26. (canceled)

27. A method for producing a composition comprising an anti-α4β7 antibody from a liquid solution comprising an anti-α4β7 antibody and one or more impurities, said method comprising
contacting the liquid solution comprising the anti-α4β7 antibody and one or more impurities with a mixed mode chromatography resin, such that the anti-α4β7 antibody binds to the resin;
washing the mixed mode chromatography resin with a wash solution; and
eluting the anti-α4β7 antibody from the mixed mode chromatography resin by contacting the resin with an elution solution having a pH at or below pH 4.2 and a conductivity at or below 28 mS/cm, such that a composition comprising the anti-α4β7 antibody is obtained,
wherein the anti-α4β7 antibody comprises a heavy chain variable region set forth in SEQ ID NO:1, and a light chain variable region set forth in SEQ ID NO:2.

28. The method of claim 27, wherein the composition comprises an increased yield of an anti-α4β7 antibody relative to a control composition comprising the anti-α4β7 antibody obtained in like manner using a control elution solution having a pH above pH 4.2 and/or a control conductivity above 28 mS/cm.

29-32. (canceled)

33. The method of claim 27, wherein:
the mixed mode chromatography resin is contacted with at least 55 g of the anti-α4β7 antibody per liter of resin; or
the mixed mode chromatography resin is contacted with about 55 g to about 80 g of the anti-α4β7 antibody per liter of resin.

34. (canceled)

35. The method of claim 27, wherein the mixed mode chromatography resin has strong anion exchange, hydrogen bonding, and hydrophobic interaction functionality on a smaller bead size, optionally wherein the mixed mode chromatography resin is Capto Adhere ImpRes.

36. The method of claim 27, wherein the method further comprises purifying the anti-α4β7 antibody using a cation exchange (CEX) resin.

37. (canceled)

38. A method for producing a composition comprising an anti-α4β7 antibody from a liquid solution comprising an anti-α4β7 antibody and one or more impurities, said method comprising
contacting the liquid solution comprising the anti-α4β7 antibody and one or more impurities with a cation exchange (CEX) resin, such that the anti-α4β7 antibody binds to the resin;
washing the CEX resin with a wash solution; and
eluting the anti-α4β7 antibody from the CEX resin by contacting the resin with an elution solution having a conductivity at or below 16 mS/cm, such that a composition comprising the anti-α4β7 antibody is obtained,
wherein the anti-α4β7 antibody comprises a heavy chain variable region set forth in SEQ ID NO:1, and a light chain variable region set forth in SEQ ID NO:2.

39. The method of claim 38, wherein the composition comprising the anti-α4β7 antibody, comprises about 1% or less HMW aggregate.

40-42. (canceled)

43. The method of claim 38, wherein the elution solution comprises NaCl at a concentration of about 70 mM to about 110 mM.

44. The method of claim 38, wherein:
the elution solution has a pH from about pH 5 to about pH 6; or
the elution solution has a pH from about pH 5.1 to about pH 5.8.

45. (canceled)

46. The method of claim 38, wherein:
the anti-α4β7 antibody is loaded on the CEX resin at a concentration of about 25-70 g antibody per liter of resin; or
the anti-α4β7 antibody is loaded on the CEX resin at a concentration of about 30-60 g antibody per liter of resin.

47-48. (canceled)

49. The method of claim 38, wherein the method further comprises purifying the anti-α4β7 antibody using a mixed mode chromatography resin.

50. (canceled)

51. A method for producing a composition comprising an anti-α4β7 antibody from a liquid solution comprising a major isoform of an anti-α4β7 antibody and one or more basic isoform species, the method comprising
contacting the liquid solution comprising the anti-α4β7 antibody and one or more basic isoform species with a cation exchange (CEX) resin, such that the anti-α4β7 antibody binds to the resin;
washing the CEX resin with a wash solution; and
eluting the anti-α4β7 antibody from the CEX resin by contacting the resin with an elution solution having a conductivity at or above 11 mS/cm, such that a composition comprising the anti-α4β7 antibody is obtained,
wherein the anti-α4β7 antibody comprises a heavy chain variable region set forth in SEQ ID NO:1, and a light chain variable region set forth in SEQ ID NO:2.

52. The method of claim 51, wherein the composition comprising the anti-α4β7 antibody, comprises about 4% to about 20% basic isoform.

53-55. (canceled)

56. The method of claim 51, wherein:
the elution solution has a pH from about pH 5 to about pH 6; or
the elution solution has a pH from about pH 5.1 to about pH 5.8.

57. (canceled)

58. The method of claim 51, wherein:
the anti-α4β7 antibody is loaded on the CEX resin at a concentration of about 25-70 g antibody per liter of resin; or
the anti-α4β7 antibody is loaded on the CEX resin at a concentration of about 30-60 g antibody per liter of resin.

59-60. (canceled)

61. The method of claim 51, wherein the method further comprises purifying the anti-α4β7 antibody using a mixed mode chromatography resin.

62. (canceled)

63. The method of claim 1, wherein the antibody was produced in a Chinese Hamster Ovary (CHO) cell.

64. (canceled)

65. The method of claim 1, wherein the composition obtained comprises purified anti-α4β7 antibody, and wherein the method further comprises a subsequent step of formulating the anti-α4β7 antibody into a formulation suitable for human use.

66-68. (canceled)

69. The method of claim 1, wherein the anti-α4β7 antibody comprises a heavy chain variable region sequence as set forth in SEQ ID NO: 1, and a light chain variable region sequence as set forth in SEQ ID NO: 5.

70. The method of claim 1, wherein the anti-α4β7 antibody is vedolizumab.

71. A composition comprising an anti-α4β7 antibody, wherein said composition is obtainable by the method of claim 1.

72. A composition comprising an anti-α4β7 antibody, wherein said composition is obtainable by the method of claim 8.

73. A composition comprising an anti-α4β7 antibody, wherein said composition is obtainable by the method of claim 17.

74. A composition comprising an anti-α4β7 antibody, wherein said composition is obtainable by the method of claim 27.

75. A composition comprising an anti-α4β7 antibody, wherein said composition is obtainable by the method of claim 38.

76. A composition comprising an anti-α4β7 antibody, wherein said composition is obtainable by the method of claim 51.


SEQUENCE LISTING (10 sequences)

SEQ ID NO: 1 - protein, 121 aa, Artificial Sequence [sequence omitted]

SEQ ID NO: 2 - protein, 5 aa, Artificial Sequence
  Description of Artificial Sequence Synthetic peptide
SYWMH

SEQ ID NO: 3 - protein, 17 aa, Artificial Sequence
  Description of Artificial Sequence Synthetic peptide
EIDPSESNTNYNQKFKG

SEQ ID NO: 4 - protein, 12 aa, Artificial Sequence
  Description of Artificial Sequence Synthetic peptide
GGYDGWDYAIDY

SEQ ID NO: 5 - protein, 112 aa, Artificial Sequence [sequence omitted]

SEQ ID NO: 6 - protein, 16 aa, Artificial Sequence
  Description of Artificial Sequence Synthetic peptide
RSSQSLAKSYGNTYLS

SEQ ID NO: 7 - protein, 7 aa, Artificial Sequence
  Description of Artificial Sequence Synthetic peptide
GISNRFS

SEQ ID NO: 8 - protein, 9 aa, Artificial Sequence
  Description of Artificial Sequence Synthetic peptide
LQGTHQPYT

SEQ ID NO: 9 - protein, 451 aa, Artificial Sequence
  Description of Artificial Sequence Synthetic polypeptide
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial Sequence
  Description of Artificial Sequence Synthetic polypeptide
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC